(19) United States
(12) Patent Application Publication
(10) Pub. No.: US 2024/0293360 A1
(43) Pub. Date: Sep. 5, 2024

(54) CRYSTALLINE ABDNAZ COMPOSITIONS AND METHODS OF MAKING AND USING THE SAME

(71) Applicant: EpicentRx, Inc., La Jolla, CA (US)

(72) Inventors: Bryan T. ORONSKY, Los Altos Hills, CA (US); Tony R. REID, San Diego, CA (US); Christpoher LARSON, San Diego, CA (US); James KANTER, La Jolla, CA (US)

(21) Appl. No.: 18/568,625

(22) PCT Filed: Jun. 9, 2022
(86) PCT No.: PCT/US2022/032780

§ 371 Date: Dec. 8, 2023

Related U.S. Application Data
(60) Provisional application No. 63/208,631, filed on Jun. 9, 2021.

(51) Int. Cl. A61K 31/397; A61K 9/14; A61K 35/14; A61K 47/10; A61P 35/00; C07D 205/04

(52) U.S. Cl. CPC A61K 31/397; A61K 9/145; A61K 35/14; A61K 47/10; A61P 35/00; C07D 205/04

(57) ABSTRACT

The present invention relates to a composition comprising solid crystalline, non-impact or non-detonation sensitive particles comprising 2-bromo-1-(3,3-dinitroazetidin-1-yl)ethanone (ABDNAZ), methods of preparing the crystalline form, and its use.

### CROSS-REFERENCE TO RELATED APPLICATIONS

This application claims priority to and the benefit of U.S. Provisional Application No. 63/208,631, filed on Jun. 9, 2021, the disclosure of which is incorporated herein by reference in its entirety.

### FIELD OF THE INVENTION

The present invention relates to a composition comprising solid crystalline, non-impact or non-detonation sensitive particles comprising 2-bromo-1-(3,3-dinitroazetidin-1-yl)ethanone (ABDNAZ), methods of preparing the crystalline form, and its use.

### BACKGROUND OF THE INVENTION

This invention relates to a new desensitized composition of ABDNAZ for use in the prevention or treatment of diseases or conditions associated with oxidative stress, inflammation, and hypoxia. As an energetic compound, which derives its intrinsically high energy value from both oxidation and ring strain, ABDNAZ possesses the highly disadvantageous property of impact sensitivity and sensitivity to detonation, which may lead to inadvertent injury or death or damage to equipment or facilities during manufacturing, transportation, formulation, and storage.

It is, therefore, an object of this invention to consistently provide a desensitized composition of ABDNAZ that is rendered inert to mechanical shock and detonation, and, hence, safer to prepare, handle, formulate, and transport. It is another object of this invention to provide a desensitized composition of ABDNAZ having improved therapeutic activity over prior art compositions. It is a further object of this invention to provide a desensitized composition of ABDNAZ having improved solubility over prior art compositions. It is yet a further object of this invention to overcome many of the handling and transport problems previously associated with impact- and detonation-sensitive materials.

Methods of synthesizing ABDNAZ have been described, such as in U.S. Pat. Nos. 7,507,842 and 8,471,041; however, the impact sensitivity and detonation sensitivity of the ABDNAZ thus synthesized are highly variable, being impossible to predict or control. Hence, the present invention provides an improved class of “desensitized” ABDNAZ, the improvements comprising inertness to shock, impact, or detonation, enhanced solubility in water and DMSO, and improved anticancer activity.

Cancer is a significant health problem despite the many advances made for detecting and treating this disease. Current strategies for managing cancer rely on early diagnosis and aggressive treatment. Treatment options often include surgery, radiotherapy, chemotherapy, hormone therapy, or a combination thereof. While such therapies provide a benefit to many patients, there is still a need for better therapeutic agents to treat various types of cancer.

Prostate cancer, breast cancer, and lung cancer are leading causes of cancer-related death. Prostate cancer is the most common form of cancer among males, with an estimated incidence of 30% in men over the age of 50. Moreover, clinical evidence indicates that human prostate cancer has the propensity to metastasize to bone, and the disease appears to progress inevitably from androgen dependent to androgen refractory status, leading to increased patient mortality. Breast cancer remains a leading cause of death in women. Its cumulative risk is relatively high; certain reports indicate that approximately one in eight women are expected to develop some type of breast cancer by age 85 in the United States. Likewise, lung cancer is a leading cause of cancer-related death, and non-small cell lung cancer (NSCLC) accounts for about 80% of these cases.

Further, inflammation, oxidative and nitrative stress, and hypoxia are hallmarks of a diverse range of diseases including cancer, ischemia reperfusion injury, autoimmunity, and trauma where effective therapy remains an unmet clinical need. ABDNAZ, as an anti-inflammatory and an anti-oxidant that is cytotoxic to tumors but not to non-malignant tissues is currently being clinically evaluated for the prevention and/or treatment of several conditions and diseases. These conditions/diseases include various cancers, ischemia reperfusion injury (IRI), and autoimmune, degenerative, and inflammatory diseases. In addition, ABDNAZ is under investigation as well as an antiradiation agent or radioprotectant to be used against nuclear radiation which may be encountered during military conflict or a nuclear meltdown and a radioprotectant and chemoprotectant intended to reduce the undesirable adverse effects of chemotherapy and radiation therapy during the treatment of cancer.

However, to recapitulate from above, ABDNAZ drug substance, despite many advantageous properties, is subject to detonation by application of shock or impact which creates potential safety issues when making, transporting, storing, and formulating the drug substance for use in therapy. Accordingly, there remains a need to create a consistently non-explosive form of ABDNAZ to enable its safe storage, transport, and general handling for use in therapeutic applications.

### SUMMARY

The present invention is based, in part, upon the discovery of a new impact- and shock- and detonation-insensitive form of ABDNAZ, its manufacture and use.

In one aspect, provided is a composition comprising solid crystalline, impact- or detonation-insensitive particles comprising the compound of Formula I:

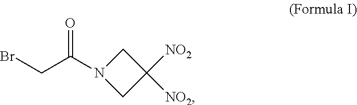 [SMILES: O=C(CBr)N1CC([N+](=O)[O-])([N+](=O)[O-])C1]

or a pharmaceutically acceptable salt thereof. In some embodiments, the composition comprises the compound in a solvated form. In some embodiments, the composition comprises tetrahydrofuran (THF). In some embodiments, the concentration of THF in the composition is at least about 330 ppm. In some embodiments, the particles are in a clathrated form. In some embodiments, the particles comprise THF. In some embodiments, the concentration of THF in the particles is at least about 330 ppm. In some embodiments, the composition further comprises n-heptane. In some embodiments, the concentration of n-heptane in the composition is at least about 800 ppm. In some embodiments, the particles comprise n-heptane. In some embodiments, the concentration of n-heptane in the particles is at least about 800 ppm.

In some embodiments, the composition has a bulk density in the range of from 0.1 g/cm³ to 0.6 g/cm³. In some embodiments, the bulk density is from 0.15 g/cm³ to 0.5 g/cm³, from 0.15 g/cm³ to 0.4 g/cm³, or from 0.16 g/cm³ to 0.3 g/cm³. In some embodiments, the particles have a bulk density less than about 0.45 g/cm³. In some embodiments, Dv(10) of the particles is less than about 40 μm. In some embodiments, Dv(50) of the particles is less than about 200 μm. In some embodiments, Dv(90) of the particles is less than about 400 μm. In some embodiments, the particles have a substantially needle-like shape. In some embodiments, the composition has a solubility greater than about 20 mg/mL in DMSO at 25° C. In some embodiments, the particles have an angle of repose of less than about 45 degrees.

In some embodiments, viability of cancer cells treated with the composition is lower than viability of cancer cells treated with an impact- or detonation-sensitive composition comprising equal amount of ABDNAZ. In some embodiments, viability of HCT 116 cells treated with about 8 μM ABDNAZ from the composition is at least about 50% lower than viability of HCT 116 cells treated with about 8 μM ABDNAZ from an impact- or detonation-sensitive composition. In some embodiments, viability of SCC VII cells treated with about 4 μM ABDNAZ from the composition is at least about 25% lower than viability of SCC VII cells treated with about 4 μM ABDNAZ from an impact- or detonation-sensitive composition. In some embodiments, viability of A549 cells treated with about 20 μM ABDNAZ from the composition is at least about 25% lower than viability of A549 cells treated with about 20 μM ABDNAZ from an impact- or detonation-sensitive composition. In some embodiments, the measurements are made about 24 hours after each treatment.

In some embodiments, viability of HCT 116 cells treated with about 10 μM ABDNAZ from the composition is less than about 25% of viability of untreated HCT 116 cells. In some embodiments, viability of SCC VII cells treated with about 4 μM ABDNAZ from the composition is less than about 50% of viability of untreated SCC VII cells. In some embodiments, viability of A549 cells treated with about 20 μM ABDNAZ from the composition is less than about 50% of viability of untreated A549 cells. In some embodiments, the measurements are made about 24 hours after each treatment.

In some embodiments, the particles are dispersed in a dedusting agent. In some embodiments, the dedusting agent is polyethylene glycol.

In some aspect, provided are pharmaceutical compositions comprising the composition described herein and a pharmaceutically acceptable carrier. In some embodiments, the pharmaceutical composition further comprises N,N-dimethylacetamide. In some embodiments, the pharmaceutical composition further comprises an anti-coagulant.

In some aspect, provided are mixtures comprising the composition described herein or the pharmaceutical composition described herein and a blood sample. In some embodiments, the blood sample is an autologous blood sample or has been harvested from a subject to be treated with the compound. In some embodiments, the concentration of the compound of Formula I is from 0.1 mg/mL of blood to 10 mg/mL of blood.

In another aspect, the invention provides a method of producing a crystalline form of the compound of Formula I, the method comprising the steps of: (a) dissolving the compound of Formula I in tetrahydrofuran, (b) adding the solution of step (a) to n-heptane with stirring, and (c) cooling the solution produced by step (b), to produce a crystalline form of Formula I. In some embodiments, the THF solution produced in step (a) is combined with the n-heptane in step (b) at a ratio of between about 1:3 (v/v) and about 1:10 (v/v). In some embodiments, during step (b), the adding occurs over a period between about 10 minutes and about 6 hours.

In one aspect, provided are crystalline forms of the compound of Formula I having the features described herein.

In some embodiments, the composition described herein is impact insensitive as determined by using a Series 3 Type (a)(ii) Test as set forth in the United Nations Manual of Tests and Criteria, seventh edition, 2019 by exposing a 40 mm³ sample of the composition to 40 J of energy. In some embodiments, the crystalline form described herein is impact insensitive as determined by using a Series 3 Type (a)(ii) Test as set forth in the United Nations Manual of Tests and Criteria, seventh edition, 2019 by exposing a 40 mm³ sample of the crystalline form to 40 J of energy.

In one aspect, provided herein are methods of treating cancer in a subject in need thereof, the method comprising administering to the subject an effective amount of the composition, the pharmaceutical composition, or the mixture described herein, thereby to treat the cancer in the subject. In another aspect, the present invention provides a method of treating cancer or protecting non-malignant tissue from damage associated with radiation and/or chemotherapeutic treatment of cancers in a subject in need thereof, the method comprising administering to the subject an effective amount of the composition described herein, the pharmaceutical composition described herein, or the mixture described herein, thereby to treat the cancer in the subject. In some embodiments, the composition or the pharmaceutical composition described herein is combined with blood harvested from the subject to create a mixture, whereupon the mixture is administered to the subject.

In one aspect, provided herein are methods of treating or preventing an ischemic or hypoxic condition in a subject in need thereof, the method comprising administering to the subject a therapeutically effective amount of the composition described herein, a pharmaceutical composition described herein, or a mixture described herein, is also provided. In some embodiments, the ischemic condition is an acute or chronic ischemic condition. In some embodiments, the acute ischemic condition is myocardial infarction, ischemic stroke, pulmonary embolism, perinatal hypoxia, circulatory shock, mountain sickness or acute respiratory failure. In some embodiments, the chronic ischemic condition is atherosclerosis, chronic venous insufficiency, chronic heart failure, cardiac cirrhosis, diabetes, macular degeneration, sleep apnea, Raynaud's disease, systemic sclerosis, nonbacterial thrombotic endocarditis, occlusive artery disease, angina pectoris, transient ischemic attacks, or chronic alcoholic liver disease. In some embodiments, the hypoxic condition is cancer, gastric or duodenal ulcers, liver or renal disease, thrombocytopenia, a blood coagulation disorder, a chronic illness, a therapeutic intervention that produces anemia such as cancer chemotherapy or altitude sickness. In some embodiments, the cancer is bladder cancer, breast cancer, clear cell kidney cancer, head/neck squamous cell carcinoma, lung squamous cell carcinoma, malignant melanoma, colorectal cancer, head and neck cancer, cervical cancer, non-small-cell lung cancer (NSCLC), ovarian cancer, pancreatic cancer, prostate cancer, renal cell cancer, small-cell lung cancer (SCLC), triple negative breast cancer, acute lymphoblastic leukemia (ALL), acute myeloid leukemia (AML), chronic lymphocytic leukemia (CLL), chronic myeloid leukemia (CML), diffuse large B-cell lymphoma (DLBCL), EBV-positive DLBCL, primary mediastinal large B-cell lymphoma, T-cell/histiocyte-rich large B-cell lymphoma, follicular lymphoma, Hodgkin's lymphoma (HL), mantle cell lymphoma (MCL), multiple myeloma (MM), myeloid cell leukemia-1 protein (Mcl-1), myelodysplasia syndrome (MDS), non-Hodgkin's lymphoma (NHL), or small lymphocytic lymphoma (SLL).

In some embodiments, the pharmaceutical composition in the method described herein comprises at least 0.5 mg of the compound of Formula I and is administered intravenously, nasally, optically, intraperitoneally, subcutaneously, or orally.

In one aspect, provided herein are methods of protecting against normal tissue toxicity caused by chemotherapy and/or radiation therapy, the method comprising: subcutaneously administering to a subject in need thereof an effective amount of the composition, the pharmaceutical composition, or the mixture described herein before the subject is exposed to the chemotherapy and/or radiation therapy. In some embodiments, the subject has cancer. In some embodiments, the cancer is head and neck cancer. In some embodiments, at least about 0.5 mg of the compound of Formula I is administered to the subject. In some embodiments, from about 0.5 mg to 4 mg of the compound of Formula I is administered to the subject. In some embodiments, the amount of the compound of Formula I is administered in one or more divided injections. In some embodiments, the normal tissue toxicity is acute mucositis or dysphagia. In some embodiments, the mucositis is late mucositis.

In one aspect, provided herein are methods of treating a disorder selected from the group consisting of an autoimmune disorder, inflammatory disorder, neurodegenerative disorder, and neuromuscular disorder in a subject in need thereof, the method comprising administering a loading dose of the composition, the pharmaceutical composition, or the mixture described herein to the subject in an amount effective to ameliorate a symptom of the disorder, and thereafter administering a maintenance dose of the composition, the pharmaceutical composition, or the mixture described herein to maintain the amelioration of the symptom for a prolonged period of time.

In one aspect, provided herein are methods for increasing compliance and tolerability in a subject in need of treatment for an autoimmune disorder, inflammatory disorder, neurodegenerative disorder, or neuromuscular disorder, the method comprising administering a therapeutically effective amount of the composition, the pharmaceutical composition, or the mixture described herein; wherein administration of the therapeutically effective amount does not cause hematologic, neurologic, pulmonary, metabolic, cardiovascular, dermatologic, nephrologic, gastrointestinal, genitourinary, inflammatory, autoimmune, thyroidal, and immunodeficiency-related side effects; and wherein the subject completes treatment with a cumulative dose of at least 1 mg or 1 mg/m² of RRx-001 or an analog thereof.

In one aspect, provided herein are methods of preventing the initiation, development or worsening of a symptom of a disorder selected from the group consisting of an autoimmune disorder, inflammatory disorder, neurodegenerative disorder, and neuromuscular disorder in a subject in need thereof, the method comprising administering an effective amount of the composition, the pharmaceutical composition, or the mixture described herein to the subject to prevent the initiation, development, or worsening of the symptom of the disorder.

In one aspect, provided herein are methods of preventing the initiation, development or worsening of a symptom of a disorder selected from the group consisting of an autoimmune disorder, inflammatory disorder, neurodegenerative disorder, and neuromuscular disorder in a subject in need thereof, the method comprising administering an effective amount of the composition, the pharmaceutical composition, or the mixture described herein to the subject to prevent the initiation, development, or worsening of the symptom of the disorder.

In one aspect, provided herein are methods for enhancing physical performance of a mammal, the method comprising: administering an effective amount of the composition, the pharmaceutical composition, or the mixture described herein to said mammal prior to said physical performance.

In one aspect, provided herein are methods for preventing or treating pulmonary hypertension (PH) in a patient, the method comprising: administering a therapeutically effective amount of the composition, the pharmaceutical composition, or the mixture described herein.

In another aspect, the present invention provides a crystalline form of the compound of Formula I produced by the methods described herein. In some embodiments, the composition can comprise the compound in a solvated form, where the solvent can be, for example, tetrahydrofuran (THF). Alternatively, or in addition, the composition can comprise the particles in a clathrated form, where the particles can comprise, for example, THF. In another aspect, the present invention provides a pharmaceutical composition comprising a composition as described herein.

### DESCRIPTION OF THE FIGURES

The present application can be understood by reference to the following description taken in conjunction with the accompanying figures.

FIGS. 1A-1C depict graphs plotting relative cell viability as a function of sample dose.

FIG. 2 depicts a graph plotting turbidity as a function of sample concentration.

FIGS. 3A and 3B depict exemplary SEM images of ABDNAZ particles with a substantially needle-like shape.

FIG. 4 depicts an exemplary SEM image of ABDNAZ particles with a substantially bulky or round shape.

FIG. 5A depicts bulk density of RRx-001 particles crystalized without THF; FIG. 5B depicts bulk density of RRx-001 particles crystalized with THF; FIG. 5C depicts bulk density of impact insensitive RRx-001 particles; and FIG. 5D depicts bulk density of impact sensitive RRx-001 particles.

FIGS. 5E and 5F depict empirical probability functions for bulk density.

FIGS. 5G and 5H depict the boxplots based on statistical analysis of bulk density.

FIG. 6A depicts D10 of RRx-001 particles crystalized without THF; FIG. 6B depicts D10 of RRx-001 particles crystalized with THF; FIG. 6C depicts D10 of impact insensitive RRx-001 particles; and FIG. 6D depicts D10 of impact sensitive RRx-001 particles.

FIGS. 6E and 6F depict empirical probability functions for D10.

FIGS. 6G and 6H depict the boxplots based on statistical analysis of D10.

FIG. 7A depicts D50 of RRx-001 particles crystalized without THF; FIG. 7B depicts D50 of RRx-001 particles crystalized with THF; FIG. 7C depicts D50 of impact insensitive RRx-001 particles; and FIG. 7D depicts D50 of impact sensitive RRx-001 particles.

FIGS. 7E and 7F depict empirical probability functions for D50.

FIGS. 7G and 7H depict the boxplots based on statistical analysis of D50.

FIG. 8A depicts D90 of RRx-001 particles crystalized without THF; FIG. 8B depicts D90 of RRx-001 particles crystalized with THF; FIG. 8C depicts D90 of impact insensitive RRx-001 particles; and FIG. 8D depicts D90 of impact sensitive RRx-001 particles.

FIGS. 8E and 8F depict empirical probability functions for D90.

FIGS. 8G and 8H depict the boxplots based on statistical analysis of D90.

### DETAILED DESCRIPTION OF THE INVENTION

The present invention provides, in part, an impact- or detonation-insensitive crystalline form of 2-bromo-1-(3,3-dinitroazetidin-1-yl)ethanone (ABDNAZ), methods of producing the crystalline form, and methods for treating various medical conditions using such compositions.

ABDNAZ, a dinitroazetidine with the formula C₅H₆BrN₃O₅, has the following chemical structure:

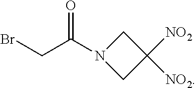 [SMILES: O=C(CBr)N1CC([N+](=O)[O-])([N+](=O)[O-])C1]

This compound is being evaluated clinically for the treatment of cancer and other ischemic/hypoxic diseases and disorders as well as protection of non-malignant tissues from the toxicities of chemotherapy and/or radiation. Prior methods of synthesizing the compound (Straessler et al. ORG. PROCESS RES. DEV. (2012) 16, 512-517) have resulted in a crystalline form of the compound. However, these crystalline forms could be explosive, and exhibit a high degree of variability with respect to impact- or detonation-sensitivity, for example, under conventional impact- or detonation-sensitivity tests.

As a result, the features of this crystalline form of the compound can impact the transportation, storage and use of the compound.

The present invention is based, in part, upon the discovery that the impact- or detonation-sensitivity of the crystalline form of the compound can be eliminated thereby making the compound safer for transport, storage and use. This has been achieved by altering or “tuning” the physiochemical characteristics of the crystal, for example, its impact- or detonation-sensitivity and flowability, without altering the intrinsic chemical properties of the composition. This tuning of the crystalline form of the compound has been achieved by implementing a particular recrystallization step at the end of synthesis using certain crystallization conditions, which assures the production of a material of normal sensitivity.

Various aspects of the invention are set forth below in sections; however, aspects of the invention described in one particular section are not to be limited to any particular section. Further, when a variable is not accompanied by a definition, the previous definition of the variable controls.

### Definitions

To facilitate an understanding of the present invention, a number of terms and phrases are defined below.

The terms “a,” “an” and “the” as used herein mean “one or more” and include the plural unless the context is inappropriate.

As used herein, the term “subject” refers to organisms to be treated by the methods of the present invention. Such organisms preferably include, but are not limited to, mammals (e.g., murines, simians, equines, bovines, porcines, canines, felines, and the like), and most preferably include humans. In the context of the invention, the term “subject” generally refers to an individual who will receive or who has received treatment (e.g., administration of a compound of the present invention and optionally one or more other agents) for a condition characterized by the dysregulation of apoptotic processes.

As used herein, the term “effective amount” refers to the amount of a compound (e.g., a compound of the present invention) sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with an excipient or a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants. (See e.g., Martin, Remington's Pharmaceutical Sciences, 15th Ed., Mack Publ. Co., Easton, PA [1975]).

As used herein, the term “pharmaceutically acceptable salt” refers to any circular salt (e.g., acid or base) of a compound of the present invention suitable for pharmaceutical administration which, upon administration to a subject, is capable of providing a compound of this invention or an active metabolite or residue thereof. As is known to those of skill in the art, “salts” of the compounds of the present invention may be derived from inorganic or organic acids and bases.

Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metals (e.g., sodium) hydroxides, alkaline earth metals (e.g., magnesium), hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

### I. Crystalline Form of 2-bromo-1-(3,3-dinitroazetidin-1-yl)ethanone (ABDNAZ)

The present invention provides, in part, an impact insensitive crystalline form of 2-bromo-1-(3,3-dinitroazetidin-1-yl)ethanone (ABDNAZ), which is a compound of Formula I:

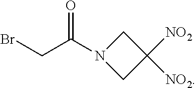 [SMILES: O=C(CBr)N1CC([N+](=O)[O-])([N+](=O)[O-])C1]

In one aspect, the present invention provides a composition comprising solid crystalline, non-impact or non-detonation sensitive particles comprising the compound of Formula I:

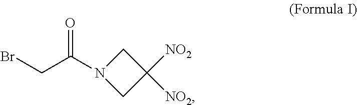 [SMILES: O=C(CBr)N1CC([N+](=O)[O-])([N+](=O)[O-])C1]

or a pharmaceutically acceptable salt thereof, the solid crystalline, non-impact or non-detonation sensitive particles having an angle of repose of less than about 45 degrees.

The composition can comprise the compound in a solvated form, where the solvent can be, for example, tetrahydrofuran (THF). In some embodiments, the solvent is any solvent or any combination of solvents described herein.

Alternatively, or in addition, the composition can comprise the particles in a clathrated form, where the particles can comprise, for example, THF. In some embodiments, the solvent is any solvent or any combination of solvents described herein.

The particles can have a combination of features described herein. For example, the particles can have a median particle size in the range from 50 μm to 300 μm, from 50 μm to 200 μm, or from 50 μm to 100 μm. The particles can have a particle size distribution wherein the Dv(10), D10, or x₁₀ is less than 50 μm, less than 40 μm, less than 30 μm, or less than 20 μm. Furthermore, the particles can have a particle size distribution wherein the Dv(50), D50, or x₅₀ value is less than 100 μm, less than 90 μm, less than 80 μm, less than 70 μm, or less than 60 μm and/or the particles can have a particle size distribution wherein the Dv(90), D90, or x₉₀ value is less than 300 μm, less than 250 μm, less than 200 μm, less than 150 μm, or less than 100 μm and/or the particles can have a particle size distribution wherein the D10 or x₁₀ value is greater than 20 μm, greater than 30 μm, greater than 40 μm, or greater than 50 μm.

In some embodiments, the impact- or detonation-insensitive ABDNAZ particles or compositions described herein have a particle size distribution where the Dv(10), D10, or x₁₀ is less than about 40 μm; the Dv(50), D50, or x₅₀ is less than about 200 μm; and/or the Dv(90), D90, or x₉₀ is less than about 400 μm. In some embodiments, the impact- or detonation-insensitive ABDNAZ particles or compositions described herein have a particle size distribution where the Dv(10), D10, or x₁₀ is less than about 5, 10, 15, 20, 25, 30, 35, 40, 45, 50, 55, 60, 65, 70, 75, or 80 μm; the Dv(50), D50, or x₅₀ is less than about 10, 15, 20, 25, 30, 35, 40, 45, 50, 55, 60, 70, 80, 90, 100, 110, 120, 130, 140, 150, 200, 250, or 300 μm; and/or the Dv(90), D90, or x₉₀ is less than about 40, 45, 50, 55, 60, 65, 70, 75, 80, 85, 90, 95, 100, 110, 120, 130, 140, 150, 160, 170, 180, 190, 200, 225, 250, 275, 300, 325, 350, 375, 400, 425, 450, 475, 500, 550, 600, 650, 700, or 800 μm.

Depending upon the recrystallization conditions, the resulting crystals can contain a residual or trace amount of tetrahydrofuran, for example, less than 800, 700, 600, 500, 400, 300, 200 or 100 ppm as determined, for example, by gas chromatography. Alternatively, or in addition, the resulting crystals may comprise a residual or trace amount of n-heptane, for example, less than 900, 800, 700, 600, 500, 400, 300, 200 or 100 ppm as determined, for example, by gas chromatography.

In some embodiments, the concentration of tetrahydrofuran in the impact- or detonation-insensitive ABDNAZ particles or compositions described herein is at least about 330 ppm. In some embodiments, the concentration of tetrahydrofuran in the impact- or detonation-insensitive ABDNAZ particles or compositions described herein is at least about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 550, 600, 650, 700, 750, 800, 850, 900, 1000, 1500, 2000, 2500, 3000, 3500, or 4000 ppm. In some embodiments, the concentration of tetrahydrofuran in the impact- or detonation-insensitive ABDNAZ particles or compositions described herein is less than about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 550, 600, 650, 700, 750, 800, 850, 900, 1000, 1500, 2000, 2500, 3000, 3500, or 4000 ppm. In some embodiments, the concentration of tetrahydrofuran in the impact- or detonation-insensitive ABDNAZ particles or compositions described herein has an upper limit of about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 550, 600, 650, 700, 750, 800, 850, 900, 1000, 1500, 2000, 2500, 3000, 3500, or 4000 ppm, and an independently selected lower limit of about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 550, 600, 650, 700, 750, 800, 850, 900, 1000, 1500, 2000, 2500, 3000, 3500, or 4000 ppm, where the upper limit is greater than the lower limit.

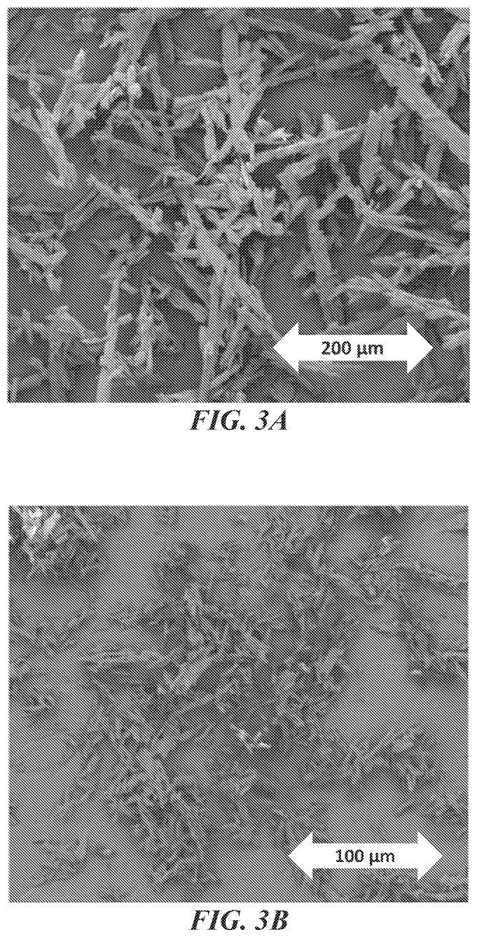

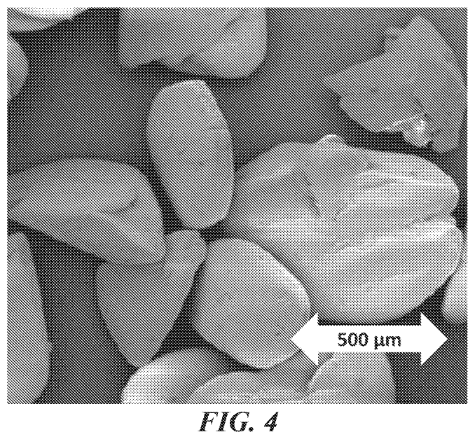

In some embodiments, the impact- or detonation-insensitive ABDNAZ particles described herein have a substantially needle-like shape. FIGS. 3A and 3B provides an example of a substantially needle-like shape. In some embodiments, the impact- or detonation-insensitive ABDNAZ particles described herein do not have a substantially bulky or round shape. FIG. 4 provides an example of a substantially bulky or round shape. In some embodiments, the shape is determined based on an SEM image.

In certain embodiments, the composition has a bulk density in the range of from 0.1 g/cm³ to 0.6 g/cm³. For example, the bulk density is from 0.15 g/cm³ to 0.5 g/cm³, from 0.15 g/cm³ to 0.4 g/cm³, or from 0.16 g/cm³ to 0.3 g/cm³.

In some embodiments, the impact- or detonation-insensitive ABDNAZ particles or compositions described herein have a bulk density less than about 0.45 g/cm³. In some embodiments, the composition has a bulk density less than about 0.2, 0.25, 0.3, 0.35, 0.4, 0.45, 0.5, 0.55, 0.6, 0.65, 0.7, 0.75, 0.8, 0.85, 0.9, 0.95, or 1 g/cm³. In some embodiments, the impact- or detonation-insensitive ABDNAZ particles or compositions described herein have a bulk density greater than about 0.2, 0.25, 0.3, 0.35, 0.4, 0.45, 0.5, 0.55, 0.6, 0.65, 0.7, 0.75, 0.8, 0.85, 0.9, 0.95, or 1 g/cm³. In some embodiments, the bulk density of the impact- or detonation-insensitive ABDNAZ particles or compositions described herein has an upper limit of about 0.2, 0.25, 0.3, 0.35, 0.4, 0.45, 0.5, 0.55, 0.6, 0.65, 0.7, 0.75, 0.8, 0.85, 0.9, 0.95, or 1 g/cm³ and an independently selected lower limit of about 0.2, 0.25, 0.3, 0.35, 0.4, 0.45, 0.5, 0.55, 0.6, 0.65, 0.7, 0.75, 0.8, 0.85, 0.9, 0.95, or 1 g/cm³, where the upper limit is greater than the lower limit.

In some embodiments, solubility or equilibrium solubility of the impact- or detonation-insensitive ABDNAZ particles or compositions described herein is greater than about 20 mg/mL in DMSO. In some embodiments, solubility or equilibrium solubility of the impact- or detonation-insensitive ABDNAZ particles or compositions described herein is greater than about 16, 17, 18, 19, 20, 21, 22, 23, 24, 25, 26, or 27 mg/mL. In some embodiments, the solubility or equilibrium solubility is measured at 25° C. In some embodiments, the solubility or equilibrium solubility is measured at 37° C. In some embodiments, the solubility or equilibrium solubility is measured in DMSO. In some embodiments, the solubility or equilibrium solubility is measured according to the methods described in N. Colclough et al., “High throughput solubility determination with application to selection of compounds for fragment screening” Bioorganic & Medicinal Chemistry Vol. 16, Issue 13 (2008): 6611-6616.

In some embodiments, viability of the cancer cells treated with the impact- or detonation-insensitive crystal or composition of ABDNAZ described herein is lower than that of the cancer cells treated with an impact- or detonation-sensitive crystal or composition of ABDNAZ. In some embodiments, the cancer cells are HCT-116, SCC VII, or A549. In some embodiments, cell viability is measured 24 hours after treatment. In some embodiments, cell viability is measured using an MTT (3-[4,5-dimethylthiazol-2-yl]-2,5-diphenyl tetrazolium bromide) colorimetric assay. In some embodiments, cell viability is measured as the optical density (OD) at 570 nm. In some embodiments, cell viability is measured relative to the control that does not receive any treatment. In some embodiments, cell viability is measured and/or compared at about 10 μM dose with HCT 116. In some embodiments, cell viability is measured and/or compared at about 4 μM dose with SCC VII. In some embodiments, cell viability is measured and/or compared at about 20 μM dose with A549. In some embodiments, cell viability is measured and/or compared at about 1, 2, 3, 4, 5, 6, 7, 8, 9, 10, 11, 12, 13, 14, 15, 16, 17, 18, 19, 20, 21, 22, 23, 24, 25, or 30 μM dose.

In some embodiments, the viability of cancer cells treated with impact- or detonation-insensitive ABDNAZ particles or composition described herein is lower than the viability of cancer cells treated with an impact- or detonation-sensitive particles or composition comprising equal amount of ABDNAZ. In some embodiments, the viability of cancer cells treated with impact- or detonation-insensitive ABDNAZ particles or composition described herein is at least about 25% lower than the viability of cancer cells treated with an impact- or detonation-sensitive particles or composition comprising equal amount of ABDNAZ. In some embodiments, the viability of cancer cells treated with impact- or detonation-insensitive ABDNAZ particles or composition described herein is at least about 5, 10, 15, 20, 25, 30, 35, 40, 45, 50, 55, 60, 65, 70, 75, 80, 85, 90, or 95% lower than the viability of cancer cells treated with an impact- or detonation-sensitive particles or composition comprising equal amount of ABDNAZ.

In some embodiments, viability of the cancer cells treated with (theoretically) about 8 μM ABDNAZ from impact- or detonation-insensitive crystal or composition described herein is at least about 50% lower than that of the cancer cells treated with (theoretically) about 8 μM ABDNAZ from impact- or detonation-sensitive crystal or composition, as measured with HCT 116 cell line 24 hours after treatment. In some embodiments, viability of the cancer cells treated with (theoretically) about 8 μM ABDNAZ from impact- or detonation-insensitive crystal or composition described herein is at least about 5, 10, 15, 20, 25, 30, 35, 40, 45, 50, 55, 60, 65, 70, 75, 80, 85, 90, or 95% lower than that of the cancer cells treated with (theoretically) about 8 μM ABDNAZ from impact- or detonation-sensitive crystal or composition, as measured with HCT 116 cell line 24 hours after treatment.

In some embodiments, “(theoretically)” in front of an ABDNAZ concentration indicates that the concentration may be based on the total amount of ABDNAZ added in a given volume, and therefore may not necessarily correspond to the concentration of ABDNAZ actually dissolved or available to the cells.

In some embodiments, viability of the cancer cells treated with (theoretically) about 4 μM ABDNAZ from impact- or detonation-insensitive crystal or composition described herein is at least about 25% lower than that of the cancer cells treated with (theoretically) about 4 μM ABDNAZ from impact- or detonation-sensitive crystal or composition, as measured with SCC VII cell line 24 hours after treatment. In some embodiments, viability of the cancer cells treated with (theoretically) about 4 μM ABDNAZ from impact- or detonation-insensitive crystal or composition described herein is at least about 5, 10, 15, 20, 25, 30, 35, 40, 45, 50, 55, 60, 65, 70, 75, 80, 85, 90, or 95% lower than that of the cancer cells treated with (theoretically) about 4 μM ABDNAZ from impact- or detonation-sensitive crystal or composition, as measured with SCC VII cell line 24 hours after treatment.

In some embodiments, viability of the cancer cells treated with (theoretically) about 20 μM ABDNAZ from impact- or detonation-insensitive crystal or composition described herein is at least about 25% lower than that of the cancer cells treated with (theoretically) about 20 μM ABDNAZ from impact- or detonation-sensitive crystal or composition, as measured with A549 cell line 24 hours after treatment. In some embodiments, viability of the cancer cells treated with (theoretically) about 20 μM ABDNAZ from impact- or detonation-insensitive crystal or composition described herein is at least about 5, 10, 15, 20, 25, 30, 35, 40, 45, 50, 55, 60, 65, 70, 75, 80, 85, 90, or 95% lower than that of the cancer cells treated with (theoretically) about 20 μM ABDNAZ from impact- or detonation-sensitive crystal or composition, as measured with A549 cell line 24 hours after treatment.

In some embodiments, viability of the cancer cells treated with (theoretically) about 10 μM ABDNAZ from impact- or detonation-insensitive crystal or composition described herein is less than about 25% of that of the control, as measured with HCT 116 cell line 24 hours after treatment. In some embodiments, viability of the cancer cells treated with (theoretically) about 10 μM ABDNAZ from impact- or detonation-insensitive crystal or composition described herein is less than about 20, 25, 30, 35, 40, or 45% of that of the control, as measured with HCT 116 cell line 24 hours after treatment.

In some embodiments, viability of the cancer cells treated with (theoretically) about 4 μM ABDNAZ from impact- or detonation-insensitive crystal or composition described herein is less than about 50% that of the control, as measured with SCC VII cell line 24 hours after treatment. In some embodiments, viability of the cancer cells treated with (theoretically) about 4 μM ABDNAZ from impact- or detonation-insensitive crystal or composition described herein is less than about 30, 35, 40, 45, 50, 55, 60, 65, or 70% of that of the control, as measured with SCC VII cell line 24 hours after treatment.

In some embodiments, viability of the cancer cells treated with (theoretically) about 20 μM ABDNAZ from impact- or detonation-insensitive crystal or composition described herein is less than about 50% that of the control, as measured with A549 cell line 24 hours after treatment. In some embodiments, viability of the cancer cells treated with (theoretically) about 20 μM ABDNAZ impact- or detonation-insensitive crystal or composition described herein is less than about 30, 35, 40, 45, 50, 55, 60, 65, or 70% of that of the control, as measured with A549 cell line 24 hours after treatment.

### II. Methods of Preparing a Crystalline Form of ABDNAZ

Methods for preparing ABDNAZ have been disclosed in U.S. Pat. No. 7,507,842 (“the '842 patent”) and U.S. Pat. No. 8,471,041 (“the '041 patent”). The method disclosed in the '842 patent involves reaction of 1-tert-butyl 3.3-dinitroazetidine (DNAZ) with bromoacetyl bromide and boron trifluoride etherate, from which ABDNAZ is isolated by cooling the reaction mixture, adding dichloromethane, filtering the DNAZ HBr that is formed, washing the dichloromethane filtrate with water, drying it, and then evaporating the dichloromethane. The method disclosed in the '041 patent involves reaction of 3,3-dinitroazetidine (DNAZ) with bromoacetyl bromide and boron trifluoride etherate in dichloromethane to produce a reaction mixture comprising ABDNAZ and a hydrogen bromide salt of DNAZ, from which the DNAZ is separated, ethanol is added to the dichloromethane and the ABDNAZ, the dichloromethane is evaporated under reduced pressure to form an ABDNAZ/ethanol suspension and the ethanol is then filtered from the ABDNAZ/ethanol suspension.

However, these patents do not address the impact- or detonation-sensitivity and sensitivity to initiation of a detonator (“detonation sensitivity”) of the obtained ABDNAZ, which are unpredictable and, therefore, dangerous.

The crystalline form of ABDNAZ that is impact- or detonation-insensitive can be produced, for example, as described herein, for example, as set forth in the following synthetic scheme.

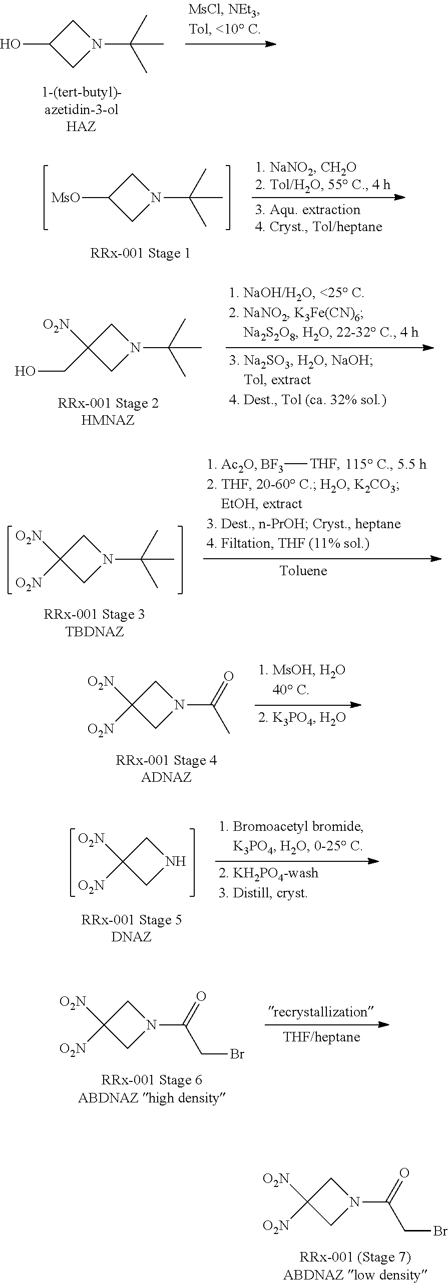

In some embodiments, HAZ is 1-tert-Butylazetidin-3-ol. In some embodiments, HMNAZ is 1-tert-butyl-3-hydroxymethyl-3-nitroazetidine. In some embodiments, TBDNAZ is 1-tert-butyl-3,3-dinitroazetidine. In some embodiments, ADNAZ is 1-acetyl-3,3-dinitroazetidine. In some embodiments, DNAZ is 3,3-dinitroazetidine.

In some embodiments, RRx-001 is synthesized according to the synthetic scheme above. In some embodiments, HAZ is methanesulfonylated to activate the alcohol for nucleophilic substitution by sodium nitrite. In some embodiments, the generated intermediate (marked “RRx-001 Stage 1” in the scheme) is trapped by formaldehyde to generate HMNAZ. In some embodiments, TBDNAZ is produced from HMNAZ by introducing the second nitro group using potassium hexacyanoferrate (III) as a catalyst. In some embodiments, the tert-butyl group of TBDNAZ is replaced by an acetyl group via a Lewis acid-catalyzed dealkylative acetylation, thus furnishing ADNAZ. In some embodiments, the acetyl group of ADNAZ is removed under acidic conditions and subsequently replaced by a bromoacetyl group using Schotten-Baumann conditions, thus generating ABDNAZ in its high density form. In some embodiments, ABDNAZ in the high density form is recrystallized to obtain its impact insensitive low density form.

RRx-001 Stages 1 and 2 (HMNAZ) Production from HAZ

In some embodiments, HAZ is methanesulfonylated. In some embodiments, methanesulfonylatioin of HAZ is to activate the alcohol for nucleophilic substitution by sodium nitrite. In some embodiments, methanesulfonyl chloride is added to HAZ for methanesulfonylatioin of HAZ. In some embodiments, water is removed prior to adding methanesulfonyl chloride to HAZ. In some embodiments, water is removed by azeotropic distillation of the starting material solution in toluene. In some embodiments, the amount of triethylamine is slightly increased to ensure that a basic pH is maintained during the following step, thus reducing the amount of NOx released. In some embodiments, the product solution is directly telescoped into the next step. In some embodiments, the reagent amount in the second step (sodium nitrite and formaldehyde) is doubled to achieve higher yields. In some embodiments, an addition time as well as stirring time of about 4 hours each are used. In some embodiments, an addition time of about 0.5, 1, 1.5, 2, 2.5, 3, 3.5, 4, 4.5, 5, 5.5, 6, 6.5, 7, 7.5, 8, 8.5, 9, 9.5, or 10 hours is used. In some embodiments, a stirring time of about 0.5, 1, 1.5, 2, 2.5, 3, 3.5, 4, 4.5, 5, 5.5, 6, 6.5, 7, 7.5, 8, 8.5, 9, 9.5, or 10 hours is used. In some embodiments, the aqueous workup is performed at elevated temperatures (e.g., about 35° C.) to keep the product in solution. In some embodiments, the elevated temperature is about 30, 32.5, 35, 37.5, 40, 42.5, 45, 47.5, or 50° C. In some embodiments, prior to concentrating the organic phase via distillation, water is added to facilitate azeotropic removal of residual formaldehyde. In some embodiments, isolation from toluene/n-heptane (˜1:1) results in pale yellow product of sufficient purity (99.8%-a/a, 99%-w/w). In some embodiments, white to off-white and analytically pure product is obtained via isolation from pure toluene at the cost of ˜4% o.th. yield.

RRx-001 Stage 3 (TBDNAZ) Production from HMNAZ

In some embodiments, terminal oxidant (sodium persulfate) for oxidative nitration is added in portions to facilitate dissolution in the reaction mixture. In some embodiments, the reaction temperature is increased to 22-32° C. to prevent RRx-001 Stage 3 (TBDNAZ) from crystallizing. In some embodiments, a quench with sodium sulfite is introduced following complete conversion to allow replacement of dichloromethane with toluene for the dilution and extraction of the product. In some embodiments, an additional aqueous wash is introduced to remove residual salts from the organic phase. In some embodiments, only azeotropic drying (instead of a solvent swap) is used when the process solvent for the following step is also toluene. In some embodiments, RRx-001 Stage 3 solution in toluene (ca. 32%-w/w) is stored under refrigerated conditions.

RRx-001 Stage 4 (ADNAZ) Production from TBDNAZ

In some embodiments, TBDNAZ and acetic anhydride are converted to ADNAZ under BF₃-catalysis at reflux. In some embodiments, TBDNAZ and acetic anhydride are converted to ADNAZ under BF₃-catalysis at reflux following a safety in-process control at 50° C. to ensure the reaction had started. In some embodiments, An upper time limit is introduced to prevent ADNAZ from decomposing. In some embodiments, dilution with THF keeps the product in solution during the quench with aqueous potassium carbonate. In some embodiments, the addition of ethanol results in superior phase separations. In some embodiments, following a solvent swap, the product is crystallized from n-propanol/n-heptane. In some embodiments, safety measures are implemented to prevent handling of impact- or detonation-sensitive material: e.g., (i) in some embodiments, the filter cake is washed water-wet, as dry ADNAZ is impact- or detonation-sensitive; and (ii) the water-wet filter cake is dissolved off the filter using THF.

### RRx-001 Stages 5 (DNAZ) and 6 (ABDNAZ “High Density”) Production

In some embodiments, removal of the acetyl group is performed under acidic conditions. In some embodiments, methanesulfonic acid is used. In some embodiments, 5 eq. of the acid and 15 eq. water is used. In some embodiments, the reaction is run at about 40° C. In some embodiments, the reaction is run at 20° C. or higher, 25° C. or higher, 30° C. or higher, 35° C. or higher, 40° C. or higher, 45° C. or higher, or 50° C. or higher. In some embodiments, the reaction mixture is quenched into aqueous potassium phosphate to avoid issues with viscosity-changes and salt-precipitation.

In some embodiments, potassium phosphate is used as the base for the following acylation. In some embodiments, Schotten-Baumann conditions are used to keep most nucleophiles into the aqueous phase, where they could not form impurities by displacing the labile α-bromide. Under these conditions, the only impurity generated stemmed from the reaction of secondary amine RRx-001 Stage 5 with the product. In some embodiments, the amount of this impurity is limited by adjusting the toluene/THF ratio (and thus the polarity of the solvent system) and/or limiting the addition time (both lower and upper limit). In some embodiments, a light acidic workup is required to guarantee the stability of RRx-001 Stage 6 in solution. In some embodiments, crystallization from toluene/MiBK (MiBK=4-methyl-2-pentanone) yields the product in API-quality. In some embodiments, due to the impact- or detonation-sensitivity of RRx-001 Stage 6, the product is dissolved from the filter using THF. In some embodiments, RRx-001 Stage 6, solution in THF needs to be stored cold.

### RRx-001 Stage 7

In some embodiments, high density ABDNAZ (dry) is dissolved in ethyl acetate. In some embodiments, the resulting solution is added to quickly stirred n-heptane. In some embodiments, the solvent is thus changed to THF. In some embodiments, to increase control over the crystallization, the solution of RRx-001 Stage 6 in THF is slowly added to quickly stirred n-heptane at an elevated temperature (e.g., 30-35° C.) and a slow cooling rate is employed.

It has been discovered that the last step in the synthesis, the recrystallization of the ABDNAZ from the Stage 6 material to the Stage 7 material is important in creating the impact- or detonation-insensitive material described herein. In this step, the protocol comprises the steps of: (a) dissolving the compound of Formula I or an impact- or detonation-sensitive ABDNAZ such as the ABDNAZ from the Stage 6 material in tetrahydrofuran, (b) adding the solution of step (a) to n-heptane at, for example, room temperature, and (c) cooling the solution of step (b) to, for example, 15° C., 10° C., or 5° C., thereby to provide the impact-insensitive crystalline form of the compound of Formula I. During step (b), the tetrahydrofuran solution is added to the heptane at a ratio of about 1:5 (v/v; i.e., volume of the THF solution:volume of heptane=1:5) over a period of at least 30 minutes, for at least, one hour, while the heptane is being stirred. During step (c) the mixture is stirred for at least 30 minutes, 45 minutes, for at least one hour. The resulting material can be harvested by filtration and dried. In some embodiments, the resulting material is impact- or detonation-sensitive when another solvent such as acetone or dichloromethane is added to heptane during step (b).

In some embodiments, the protocol comprises step (a) dissolving the compound of Formula I or an impact- or detonation-sensitive ABDNAZ such as the ABDNAZ from the Stage 6 material in solvent 1. In some embodiments, the protocol comprises step (b) adding the solution of step (a) to solvent 2 at temperature T₁. In some embodiments, the protocol comprises step (c) cooling the solution of step (b) from temperature T₁ to T₂, thereby providing the impact-insensitive crystalline form of the compound of Formula I. In some embodiments of step (b), the solution of step (a) is added to solvent 2 at a ratio of about 1:R₁ (v/v; volume of the THF solution:volume of heptane=1:R₁). In some embodiments of step (b), the solution of step (a) is added to solvent 2 over a period of t₁, while solvent 2 is being stirred. In some embodiments of step (c), the mixture is stirred for a period of t₂. In some embodiments, the resulting material can be harvested by filtration and dried.

In some embodiments, solvent 1 is a polar aprotic solvent. In some embodiments, solvent 1 is a borderline polar aprotic solvent. In some embodiments, the solvent has a dielectric constant less than about 10. In some embodiments, the solvent has a dielectric constant less than about 7, 7.5, 8, 8.5, 9, 9.5, 10, 11, 12, 13, 14, 15, 20, 30, 40, or 50. In some embodiments, the solvent has a dipole moment less than about 2. In some embodiments, the solvent has a dipole moment less than about 1.7, 1.8, 1.9, 2, 2.5, 3, 3.5, 4, 4.5, 5, or 5.5. In some embodiments, the solvent is tetrahydrofuran, dichloromethane, ethyl acetate, or any combination thereof. In some embodiments, the solvent is tetrahydrofuran. In some embodiments, solvent 1 is an oxygenated solvent. In some embodiments, solvent 1 is an alcohol, ester, or ketone. In some embodiments, solvent 1 is ethyl acetate (EtOAc), methyl isobutyl ketone (MiBK), tetrahydrofuran (THF), dichloromethane, or any combination thereof. In some embodiments, solvent 1 is acetic acid, ethyl acetate, diethyl ether, methylene chloride, n-butyl acetate, chlorobenzene, o-dichlorobenzene, or any combination thereof.

In some embodiments, solvent 2 is a non-polar solvent. In some embodiments, solvent 2 is a polar solvent. In some embodiments, solvent 2 is n-heptane, methyl isobutyl ketone (MiBK), dichloromethane, or any combination thereof.

In some embodiments, solvent 1 is EtOAc and solvent 2 is n-heptane. In some embodiments, solvent 1 is ethanol and solvent 2 is dichloromethane. In some embodiments, solvent 1 is MiBK and solvent 2 is n-heptane. In some embodiments, solvent 1 is THF and solvent 2 is n-heptane.

In some embodiments, the concentration of solvent 1 in the impact- or detonation-insensitive ABDNAZ particles or compositions described herein is at least about 330 ppm. In some embodiments, the concentration of solvent 1 in the impact- or detonation-insensitive ABDNAZ particles or compositions described herein is at least about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 550, 600, 650, 700, 750, 800, 850, 900, 1000, 1500, 2000, 2500, 3000, 3500, or 4000 ppm. In some embodiments, the concentration of solvent 1 in the impact- or detonation-insensitive ABDNAZ particles or compositions described herein is less than about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 550, 600, 650, 700, 750, 800, 850, 900, 1000, 1500, 2000, 2500, 3000, 3500, or 4000 ppm. In some embodiments, the concentration of solvent 1 in the impact- or detonation-insensitive ABDNAZ particles or compositions described herein has an upper limit of about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 550, 600, 650, 700, 750, 800, 850, 900, 1000, 1500, 2000, 2500, 3000, 3500, or 4000 ppm, and an independently selected lower limit of about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 550, 600, 650, 700, 750, 800, 850, 900, 1000, 1500, 2000, 2500, 3000, 3500, or 4000 ppm, where the upper limit is greater than the lower limit.

In some embodiments, the concentration of solvent 2 in the impact- or detonation-insensitive ABDNAZ particles or compositions described herein is at least about 800 ppm. In some embodiments, the concentration of solvent 2 in the impact- or detonation-insensitive ABDNAZ particles or compositions described herein is at least about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 550, 600, 650, 700, 750, 800, 850, 900, 1000, 1100, 1200, 1300, 1400, 1500, 1600, 1700, 1800, 1900, 2000, 2500, 3000, 3500, 4000, 4500, or 5000 ppm. In some embodiments, the concentration of solvent 2 in the impact- or detonation-insensitive ABDNAZ particles or compositions described herein is less than about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 550, 600, 650, 700, 750, 800, 850, 900, 1000, 1100, 1200, 1300, 1400, 1500, 1600, 1700, 1800, 1900, 2000, 2500, 3000, 3500, 4000, 4500, or 5000 ppm. In some embodiments, the concentration of solvent 2 in the impact- or detonation-insensitive ABDNAZ particles or compositions described herein has an upper limit of about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 550, 600, 650, 700, 750, 800, 850, 900, 1000, 1100, 1200, 1300, 1400, 1500, 1600, 1700, 1800, 1900, 2000, 2500, 3000, 3500, 4000, 4500, or 5000 ppm, and an independently selected lower limit of about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 550, 600, 650, 700, 750, 800, 850, 900, 1000, 1100, 1200, 1300, 1400, 1500, 1600, 1700, 1800, 1900, 2000, 2500, 3000, 3500, 4000, 4500, or 5000 ppm, where the upper limit is greater than the lower limit.

In some embodiments, T₁ is between about 30 and about 35° C. In some embodiments, T₁ is at least 20° C. In some embodiments, T₁ is about 15, 16, 17, 18, 19, 20, 21, 22, 23, 24, 25, 26, 27, 28, 29, 30, 32.5, 35, 37.5, 40, 42.5, 45, 47.5, 50, 55, 60, 65, 70, 75, 80, 85, 90, or 95° C. In some embodiments, T₁ is at least about 15, 16, 17, 18, 19, 20, 21, 22, 23, 24, 25, 26, 27, 28, 29, 30, 32.5, 35, 37.5, 40, 42.5, 45, 47.5, 50, 55, 60, 65, 70, 75, 80, 85, 90, or 95° C. In some embodiments, T₁ is less than about 15, 16, 17, 18, 19, 20, 21, 22, 23, 24, 25, 26, 27, 28, 29, 30, 32.5, 35, 37.5, 40, 42.5, 45, 47.5, 50, 55, 60, 65, 70, 75, 80, 85, 90, or 95° C. In some embodiments, T₁ has an upper limit of about 15, 16, 17, 18, 19, 20, 21, 22, 23, 24, 25, 26, 27, 28, 29, 30, 32.5, 35, 37.5, 40, 42.5, 45, 47.5, 50, 55, 60, 65, 70, 75, 80, 85, 90, or 95° C., and an independently selected lower limit of about 15, 16, 17, 18, 19, 20, 21, 22, 23, 24, 25, 26, 27, 28, 29, 30, 32.5, 35, 37.5, 40, 42.5, 45, 47.5, 50, 55, 60, 65, 70, 75, 80, 85, 90, or 95° C., where the upper limit is greater than the lower limit.

In some embodiments, T₂ is between about 0 and about 10° C. In some embodiments, T₂ is less than about 20° C. In some embodiments, T₂ is about −20, −17.5, −15, −12.5, −10, −7.5, −5, −2.5, 0, 2.5, 5, 7.5, 10, 12.5, 15, 17.5, 20, 22.5, 25, 27.5, 30, 32.5, 35, 37.5, 40, 42.5, 45, 47.5, 50, 55, 60, 65, 70, 75, 80, 85, or 90° C. In some embodiments, T₂ is at least about −20, −17.5, −15, −12.5, −10, −7.5, −5, −2.5, 0, 2.5, 5, 7.5, 10, 12.5, 15, 17.5, 20, 22.5, 25, 27.5, 30, 32.5, 35, 37.5, 40, 42.5, 45, 47.5, 50, 55, 60, 65, 70, 75, 80, 85, or 90° C. In some embodiments, T₂ is less than about −20, −17.5, −15, −12.5, −10, −7.5, −5, −2.5, 0, 2.5, 5, 7.5, 10, 12.5, 15, 17.5, 20, 22.5, 25, 27.5, 30, 32.5, 35, 37.5, 40, 42.5, 45, 47.5, 50, 55, 60, 65, 70, 75, 80, 85, or 90° C. In some embodiments, T₂ has an upper limit of about −20, −17.5, −15, −12.5, −10, −7.5, −5, −2.5, 0, 2.5, 5, 7.5, 10, 12.5, 15, 17.5, 20, 22.5, 25, 27.5, 30, 32.5, 35, 37.5, 40, 42.5, 45, 47.5, 50, 55, 60, 65, 70, 75, 80, 85, or 90° C., and an independently selected lower limit of about −20, −17.5, −15, −12.5, −10, −7.5, −5, −2.5, 0, 2.5, 5, 7.5, 10, 12.5, 15, 17.5, 20, 22.5, 25, 27.5, 30, 32.5, 35, 37.5, 40, 42.5, 45, 47.5, 50, 55, 60, 65, 70, 75, 80, 85, or 90° C., where the upper limit is greater than the lower limit.

In some embodiments, R₁ is about 5. In some embodiments, R₁ is between about 3 and about 10. In some embodiments, R₁ is about 0.1, 0.2, 0.3, 0.4, 0.5, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 25, 30, 35, 40, 45, or 50. In some embodiments, R₁ is at least about 0.1, 0.2, 0.3, 0.4, 0.5, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 25, 30, 35, 40, 45, or 50. In some embodiments, R₁ is less than about 0.1, 0.2, 0.3, 0.4, 0.5, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 25, 30, 35, 40, 45, or 50. In some embodiments, R₁ has an upper limit of about 0.1, 0.2, 0.3, 0.4, 0.5, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 25, 30, 35, 40, 45, or 50, and an independently selected lower limit of about 0.1, 0.2, 0.3, 0.4, 0.5, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 25, 30, 35, 40, 45, or 50, where the upper limit is greater than the lower limit.

In some embodiments, t₁ is about 0.5 hour. In some embodiments, t₁ is between about 10 minutes and about 6 hours. In some embodiments, t₁ is at least about 10 minutes. In some embodiments, t₁ is about 0.1, 0.2, 0.3, 0.4, 0.5, 0.6, 0.7, 0.8, 0.9, 1, 1.25, 1.5, 1.75, 2, 2.25, 2.5, 2.75, 3, 3.5, 4, 4.5, 5, 6, 7, 8, 9, or 10 hours. In some embodiments, t₁ is at least about 1, 2, 3, 4, 5, 6, 7, 8, 9, 10, 11, 12, 13, 14, 15, 16, 17, 18, 19, 20, 25, 30, 35, 40, 45, 50, 55, 60, 70, 80, 90, 100, 110, 120, 130, 140, 150, 160, 170, 180, 210, or 240 minutes. In some embodiments, t₁ is less than about 0.1, 0.2, 0.3, 0.4, 0.5, 0.6, 0.7, 0.8, 0.9, 1, 1.25, 1.5, 1.75, 2, 2.25, 2.5, 2.75, 3, 3.5, 4, 4.5, 5, 6, 7, 8, 9, or 10 hours. In some embodiments, t₁ has an upper limit of about 0.1, 0.2, 0.3, 0.4, 0.5, 0.6, 0.7, 0.8, 0.9, 1, 1.25, 1.5, 1.75, 2, 2.25, 2.5, 2.75, 3, 3.5, 4, 4.5, 5, 6, 7, 8, 9, or 10 hours, and an independently selected lower limit of about 1, 2, 3, 4, 5, 6, 7, 8, 9, 10, 11, 12, 13, 14, 15, 16, 17, 18, 19, 20, 25, 30, 35, 40, 45, 50, 55, 60, 70, 80, 90, 100, 110, 120, 130, 140, 150, 160, 170, 180, 210, or 240 minutes, where the upper limit is longer than the lower limit.

In some embodiments, t₂ is at least about 2 hours. In some embodiments, t₂ is at least about 0.5 hour. In some embodiments, t₂ is about 0.1, 0.2, 0.3, 0.4, 0.5, 0.6, 0.7, 0.8, 0.9, 1, 1.25, 1.5, 1.75, 2, 2.25, 2.5, 2.75, 3, 3.5, 4, 4.5, 5, 6, 7, 8, 9, or 10 hours. In some embodiments, t₂ is at least about 0.1, 0.2, 0.3, 0.4, 0.5, 0.6, 0.7, 0.8, 0.9, 1, 1.25, 1.5, 1.75, 2, 2.25, 2.5, 2.75, 3, 3.5, 4, 4.5, 5, 6, 7, 8, 9, or 10 hours. In some embodiments, t₂ is less than about 0.1, 0.2, 0.3, 0.4, 0.5, 0.6, 0.7, 0.8, 0.9, 1, 1.25, 1.5, 1.75, 2, 2.25, 2.5, 2.75, 3, 3.5, 4, 4.5, 5, 6, 7, 8, 9, or 10 hours. In some embodiments, t₂ has an upper limit of about 0.1, 0.2, 0.3, 0.4, 0.5, 0.6, 0.7, 0.8, 0.9, 1, 1.25, 1.5, 1.75, 2, 2.25, 2.5, 2.75, 3, 3.5, 4, 4.5, 5, 6, 7, 8, 9, or 10 hours, and an independently selected lower limit of about 0.1, 0.2, 0.3, 0.4, 0.5, 0.6, 0.7, 0.8, 0.9, 1, 1.25, 1.5, 1.75, 2, 2.25, 2.5, 2.75, 3, 3.5, 4, 4.5, 5, 6, 7, 8, 9, or 10 hours, where the upper limit is greater than the lower limit.

The resulting compound can be tested for impact- or detonation-sensitivity using one or more standard protocols, for example, as described in the United Nations Manual of Tests and Criteria, seventh edition, 2019 (Orange Book). For example, the explosiveness of a compound, for example, crystalline ABDNAZ, can be determined by a Series 3 Type (a)(ii) Test procedure using a BAM Fallhammer, as described in the Orange Book. Tests are conducted such that 40 mm³ of sample disposed in an impact device is subjected to 40 J of energy (e.g., using a 10 kg drop weight dropped from a height of 40 cm). The test is conducted on six separate samples under the same experimental conditions, and the operator determines whether an explosion has occurred. If no explosion occurs on all six samples, then the material is characterized as not impact- or detonation-sensitive or impact- or detonation-insensitive. However, if one of the samples fails the test and causes an explosion then the material is characterized as impact- or detonation-sensitive.

With previous compositions of RRx-001, positive results were observed with the BAM Fallhammer test at 40 Joules (10 kg mass at 0.4 m height), which is comparable to those of the explosives TNT, HMX, and RDX. On this basis, RRx-001 has been categorized as a Class 1 explosive and thus specially equipped and permitted facilities, preferably distant from dense population centers and conforming to the most up-to-date explosive safety and environmental regulations, are required as well as personnel that are duly trained and certified to synthesize and store these compounds. For clinical use, only a handful of facilities with the proper CMC/cGMP expertise and equipment exist globally. Thus, the large-scale manufacturing of prior compositions of RRx-001 is constrained to niche contract manufacturing organizations (CMOs) with the capability and experience to provide late-stage clinical material and be a viable option for commercialization. However, with the composition disclosed by this invention, a consistently and repeatedly negative BAM Fallhammer test result with no reaction was and is observed (10 kg mass, 0.40 m height, 40 J).

Another standard test evaluates whether a material can be caused to detonate with a number 8 blasting cap. A “number 8 test blasting cap” refers to a metallic capsule which contains an initiating explosive charge used to detonate the test material, which in the case is ABDNAZ. With the aforementioned compositions of ABDNAZ, the detonation sensitivity was variable, that is, sometimes the ABDNAZ was able to be detonated and sometimes the ABDNAZ was not able to be detonated.

### III. Pharmaceutical Compositions

The invention provides pharmaceutical compositions comprising an active therapeutic agent and one or more pharmaceutically acceptable carriers (additives) and/or diluents. In certain embodiments, the active therapeutic agent is ABDNAZ, such that the invention provides a pharmaceutical composition comprising ABDNAZ formulated together with one or more pharmaceutically acceptable carriers (additives) and/or diluents. The pharmaceutical composition may comprise ABDNAZ in a therapeutically effective amount. As described in detail below, the pharmaceutical compositions of the present invention may be specially formulated for administration in liquid form, including those adapted for the following: (1) oral administration, for example, drenches (aqueous or non-aqueous solutions or suspensions); and (2) parenteral administration by, for example, subcutaneous, intramuscular, intravenous or epidural injection as, for example, a sterile solution or suspension, or sustained-release formulation.

The phrase “therapeutically-effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The amount of active ingredient which can be combined with a carrier material to produce a single dosage form will generally be that amount of the compound which produces a therapeutic effect. In some embodiments, out of one hundred percent, this amount will range from about 0.1 percent to about ninety-nine percent of active ingredient, preferably from about 5 percent to about 70 percent, most preferably from about 10 percent to about 30 percent.

In some embodiments, water is a vehicle when the pharmaceutical compositions of the present invention is administered intravenously. In some embodiments, saline solutions and aqueous dextrose and glycerol solutions can also be employed as liquid vehicles, particularly for injectable solutions. In some embodiments, suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

In some embodiments, the present pharmaceutical compositions, if desired, can also contain minor amounts of wetting or emulsifying agents, or pH buffering agents. In addition, auxiliary, stabilizing, thickening, lubricating and coloring agents may be used.

In some embodiments, any of the pharmaceutical compositions described herein, may be manufactured by means of conventional mixing, dissolving, granulating, dragee-making, levigating, emulsifying, encapsulating, entrapping or lyophilizing processes. In some embodiments, pharmaceutical compositions may be formulated in conventional manner using one or more physiologically acceptable carriers, diluents, excipients or auxiliaries, which facilitate processing of compounds into preparations which can be used pharmaceutically. Proper formulation is dependent upon the route of administration chosen.

In some embodiments, the pharmaceutical compositions of the present invention are formulated in accordance with routine procedures as a pharmaceutical composition adapted for intravenous administration to human beings. In some embodiments, the pharmaceutical compositions of the present invention are solutions in sterile isotonic aqueous buffer for intravenous administration. In some embodiments, for injection, any of the compositions described herein may be formulated in aqueous solutions, e.g., in physiologically compatible buffers such as Hanks' solution, Ringer's solution, or physiological saline buffer. In some embodiments, the solution may contain formulatory agents such as suspending, stabilizing and/or dispersing agents. In some embodiments, when necessary, the pharmaceutical compositions may also include a solubilizing agent. In some embodiments, the pharmaceutical compositions for intravenous administration may optionally include a local anesthetic such as lignocaine to ease pain at the site of the injection. In some embodiments, the ingredients are supplied either separately or mixed together in unit dosage form, for example, as a lyophilized powder or water free concentrate in a hermetically sealed container such as an ampoule or sachette indicating the quantity of active agent. In some embodiments, when the pharmaceutical compositions of the present invention are administered by infusion, it can be dispensed, for example, with an infusion bottle containing sterile pharmaceutical grade water or saline. In other embodiments, when the pharmaceutical compositions of the present invention are administered by injection, an ampoule of sterile water for injection or saline can be provided so that the ingredients may be mixed prior to administration.

In some embodiments, liquid dosage forms for oral administration of the compounds of the invention include pharmaceutically acceptable emulsions, microemulsions, solutions, aqueous or oily suspensions, syrups and elixirs. In some embodiments, in addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

In some embodiments, besides inert diluents, the oral compositions can also include adjuvants such as wetting agents, emulsifying and suspending agents, sweetening, flavoring, coloring, perfuming and preservative agents.

In some embodiments, suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

In some embodiments, pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. In some embodiments, proper fluidity can be maintained, for example, by the use of coating materials, such as lecithin, by the maintenance of the required particle size in the case of dispersions, and by the use of surfactants.

In some embodiments, these compositions may also contain adjuvants such as preservatives, wetting agents, emulsifying agents and dispersing agents. In some embodiments, prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In other embodiments, prolonged absorption of the injectable pharmaceutical form may be brought about by the inclusion of agents which delay absorption such as aluminum monostearate and gelatin. In some embodiments, liquid drug formulations suitable for use with nebulizers and liquid spray devices and EHD aerosol devices typically include the composition with a pharmaceutically acceptable vehicle. In some embodiment, the pharmaceutically acceptable vehicle is a liquid such as alcohol, water, polyethylene glycol or a perfluorocarbon. Optionally, another material may be added to alter the aerosol properties of the solution or suspension of compounds. In some embodiments, this material is liquid such as an alcohol, glycol, polyglycol or a fatty acid. Other methods of formulating liquid drug solutions or suspension suitable for use in aerosol devices are known to those of skill in the art (see, e.g., Biesalski, U.S. Pat. No. 5,112,598; Biesalski, U.S. Pat. No. 5,556,611).

In some embodiments, the preparations of the present invention may be given, for example, orally or parenterally. The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

In some embodiments, systemic formulations include those designed for administration by injection, e.g., subcutaneous, intravenous, intramuscular, intrathecal or intraperitoneal injection, as well as those designed for transdermal, transmucosal, oral or pulmonary administration. Systemic formulations may be made in combination with a further active agent that improves mucociliary clearance of airway mucus or reduces mucous viscosity. These active agents include, but are not limited to, sodium channel blockers, antibiotics, N-acetyl cysteine, homocysteine and phospholipids.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

in some embodiments, the compositions described herein may be administered to humans and other animals for therapy by any suitable route of administration, including orally, nasally, as by, for example, a spray, rectally, intravaginally, parenterally, intracisternally and topically.

In some embodiments, for topical administration, the compositions described in the current application may be formulated as by powders, ointments or drops, including buccally and sublingually, solutions, gels, ointments, creams, suspensions, etc. as is well-known in the art.

In some embodiments, for buccal administration, the pharmaceutical compositions may take the form of tablets, lozenges, etc. formulated in conventional manner.

In some embodiments, for transmucosal administration, penetrants appropriate to the barrier to be permeated are used in the formulation. Such penetrants are generally known in the art.

In some embodiments, the present pharmaceutical compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable vehicle is a capsule (e.g., Grosswald et al., U.S. Pat. No. 5,698,155). A general discussion of the preparation of pharmaceutical compositions may be found in Remington, “The Science and Practice of Pharmacy,” 19th Edition.

In other embodiments, the present pharmaceutical compositions, when in capsule, tablet or pill form, may be coated to delay disintegration and absorption in the gastrointestinal tract, thereby providing a sustained action over an extended period of time. In some embodiments, selectively permeable membranes surrounding an osmotically active driving compound are also suitable for orally administered compounds. In some embodiments, in these later platforms, fluid from the environment surrounding the capsule is imbibed by the driving compound, which swells to displace the agent or agent composition through an aperture. In some embodiments, these delivery platforms can provide an essentially zero order delivery profile as opposed to the spiked profiles of immediate release formulations. In some embodiments, a time delay material such as glycerol monostearate or glycerol stearate may also be used. In some embodiments, oral compositions can include standard vehicles such as mannitol, lactose, starch, magnesium stearate, sodium saccharine, cellulose, magnesium carbonate, etc. In some embodiments, such vehicles are of pharmaceutical grade.

In some embodiments, the crystalline form of ABDNAZ described herein and/or pharmaceutical compositions thereof may also be formulated in rectal or vaginal pharmaceutical compositions such as suppositories or retention enemas, e.g., containing conventional suppository bases such as cocoa butter or other glycerides.

In some embodiments, actual dosage levels of the active ingredients in the pharmaceutical compositions of this invention may be varied so as to obtain an amount of the active ingredient Compound 1 which is effective to achieve the desired therapeutic response for a particular patient, composition, and mode of administration, without being toxic to the patient.

In some embodiments, the selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the composition of the invention employed in the pharmaceutical composition at levels lower than that required in order to achieve the desired therapeutic effect and gradually increase the dosage until the desired effect is achieved.

In some embodiments, a suitable daily dose of a composition of the invention will be that amount of the compound which is the lowest dose effective to produce a therapeutic effect. Such an effective dose will generally depend upon the factors described above. In some embodiments, the compounds are administered at about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone.

In some embodiments, the effective daily dose of the active compound may be administered as two, three, four, five, six or more sub-doses administered separately at appropriate intervals throughout the day, optionally, in unit dosage forms. In some embodiments, dosing is one administration per day.

The description above describes multiple aspects and embodiments of the invention. The patent application specifically contemplates all combinations and permutations of the aspects and embodiments.

### IV. Therapeutic Administration

When used to treat or prevent the above disease or disorders, the compositions disclosed herein may be administered or applied singly, or in combination with other agents. ABDNAZ and/or pharmaceutical compositions thereof may also be administered or applied singly, or in combination with other pharmaceutically active agents (e.g., other anti-cancer agents, other arthritis agents, etc.).

The compositions disclosed herein and another therapeutic agent can act additively or synergistically. In one embodiment, the crystalline form of ABDNAZ disclosed herein and/or a pharmaceutical composition thereof are administered concurrently with the administration of another therapeutic agent. In another embodiment, a formulation and/or pharmaceutical composition of the crystalline form of ABDNAZ thereof is administered prior or subsequent to administration of another therapeutic agent.

Sequential or concurrent administration of each therapeutic agent can be effected by any appropriate route including, but not limited to, oral routes, intravenous routes, intramuscular routes, and direct absorption through mucous membrane tissues.

It is understood that the therapeutic agents can be administered by the same route or by different routes. In some embodiments, a first therapeutic agent of the combination selected may be administered by intravenous administration while the other therapeutic agent(s) of the combination may be administered orally. Alternatively, for example, all therapeutic agents may be administered orally or all therapeutic agents may be administered by intravenous injection.

In particular, in one embodiment, the crystalline form of ABDNAZ disclosed herein and/or pharmaceutical compositions thereof can be used in combination therapy with other chemotherapeutic agents (e.g., alkylating agents (e.g., nitrogen mustards (e.g., cyclophosphamide, ifosfamide, mechlorethamine, melphalen, chlorambucil, hexamethylmelamine, thiotepa), alkyl sulfonates (e.g., busulfan), nitrosoureas, triazines)), antimetabolites (e.g., folic acid analogs, pyrimidine analogs (e.g., fluorouracil, floxuridine, cytosine arabinoside, etc.), purine analogs (e.g., mercaptopurine, thiogunaine, pentostatin, etc.), natural products (e.g., vinblastine, vincristine, etoposide, tertiposide, dactinomycin, daunorubicin, doxurubicin, bleomycin, mithrmycin, mitomycin C, L-asparaginase, interferon alpha), platinum coordination complexes (e.g., cis-platinum, carboplatin, etc.), apoptosis inducing agents, glutathione depleting agents or other agents that can alter the redox status of the cell. Those of skill in the art will appreciate that the compositions disclosed herein may also be used in concurrent combination therapy with both the chemotherapeutic agents listed above and radiotherapy.

In some embodiments, the compositions disclosed herein may be administered orally. The compositions disclosed herein may also be administered by any other convenient route, for example, by infusion or bolus injection, by absorption through epithelial or mucocutaneous linings (e.g., oral mucosa, rectal and intestinal mucosa, etc.). Administration can be systemic or local. Various delivery systems are known, (e.g., encapsulation in liposomes, microparticles, microcapsules, capsules, etc.) that can be used to administer the compositions disclosed herein. Methods of administration include, but are not limited to, intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes or skin. The mode of administration is left to the discretion of the practitioner and will depend in-part upon the site of the medical condition. In most instances, administration will result in the release of ABDNAZ and/or pharmaceutical compositions thereof into the bloodstream.

In some embodiments, the compositions disclosed herein may be administered via a medical device, for example, using a medication infusion device, system, and methods as described in International Publication No. WO 2019/241276, the entire disclosure of which is incorporated herein.

In specific embodiments, it may be desirable to administer the compositions disclosed herein locally to the area in need of treatment. This may be achieved, for example, and not by way of limitation, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes or fibers. In one embodiment, administration can be by direct injection at the site (or former site) of the disease or disorder.

In certain embodiments, it may be desirable to introduce the compositions disclosed herein into the central nervous system by any suitable route, including intraventricular, intrathecal and epidural injection. Intraventricular injection may be facilitated by an intraventricular catheter, for example, attached to a reservoir, such as an Ommaya reservoir.

In other embodiments, the compositions disclosed herein may also be administered directly to the lung by inhalation. For administration by inhalation, ABDNAZ and/or pharmaceutical composition thereof may be conveniently delivered to the lung by a number of different devices. For example, a Metered Dose Inhaler (“MDI”), which utilizes canisters that contain a suitable low boiling propellant, (e.g., dichlorodifluoromethane, trichlorofluoromethane, dichlorotetrafluoroethane, carbon dioxide or any other suitable gas) may be used to deliver ABDNAZ and/or pharmaceutical compositions thereof directly to the lung.

In another embodiment, a Dry Powder Inhaler (“DPI”) device may be used to administer the compositions disclosed herein to the lung. DPI devices typically use a mechanism such as a burst of gas to create a cloud of dry powder inside a container, which may then be inhaled by the patient and are well known in the art. In a particular embodiment, a popular variation is the multiple dose DPI (“MDDPI”) system, which allows for the delivery of more than one therapeutic dose. MDDPI devices are commercially available from a number of pharmaceutical companies e.g., Schering Plough, Madison, NJ). For example, capsules and cartridges of gelatin for use in an inhaler or insufflator may be formulated containing a powder mix of the compositions disclosed herein and a suitable powder base such as lactose or starch for these systems.

In some embodiments, another type of device that may be used to deliver the compositions disclosed herein to the lung is a liquid spray device supplied, for example, by Aradigm Corporation, Hayward, CA. Liquid spray systems use extremely small nozzle holes to aerosolize liquid drug formulations that may then be directly inhaled into the lung.

In some embodiments, a nebulizer is used to deliver the compositions disclosed herein to the lung. Nebulizers create aerosols from liquid drug formulations by using, for example, ultrasonic energy to form fine particles that may be readily inhaled (see e.g., Verschoyle et al., British J. Cancer, 1999, 80, Suppl. 2, 96). Examples of nebulizers include devices supplied by Sheffield Pharmaceuticals, St. Louis, MO. (Armer et al., U.S. Pat. No. 5,954,047; van der Linden el al., U.S. Pat. No. 5,950,619; van der Linden et al., U.S. Pat. No. 5,970,974) and Batelle Pulmonary Therapeutics, Columbus, OH).

In other embodiments, an electrohydrodynamic (“EHD”) aerosol device is used to deliver the compositions disclosed herein to the lung of a patient. EHD aerosol devices use electrical energy to aerosolize liquid drug solutions or suspensions (see e.g., Noakes et al., U.S. Pat. No. 4,765,539). The electrochemical properties of the formulation may be important parameters to optimize when delivering ABDNAZ and/or pharmaceutical composition thereof to the lung with an EHD aerosol device. EHD aerosol devices may more efficiently deliver drugs to the lung than existing pulmonary delivery technologies.

In some embodiments, the compositions disclosed herein can be delivered in a vesicle, in particular a liposome (e.g., Langer, 1990, Science, 249:1527-1533; Treat et al., in “Liposomes in the Therapy of Infectious Disease and Cancer,” Lopez-Berestein and Fidler (eds.), Liss, New York, pp. 353-365 (1989)).

In some embodiments, the compositions disclosed herein can be delivered via sustained release systems, e.g., oral sustained release systems. In other embodiments, a pump may be used (e.g., Langer, supra, Sefton, 1987, CRC Crit. Ref Biomed. Eng. 14:201; Saudek el al., 1989, N. Engl. J Med. 321:574).

In some embodiments, polymeric materials can be used (e.g., “Medical Applications of Controlled Release,” Langer and Wise (eds.), CRC Press, Boca Raton, Florida (1974); “Controlled Drug Bioavailability,” Drug Product Design and Performance, Smolen and Ball (eds.), Wiley, New York (1984); Ranger et al., 1983, J Macromol. Sci. Rev. Macromol Chem. 23:61; Levy et al., 1985, Science 228: 190; During et al., 1989, Ann. Neurol. 25:351; Howard el al., 1989, J. Neurosurg. 71:105).

In other embodiments, polymeric materials are used for oral sustained release delivery. Polymers include, but are not limited to, sodium carboxymethylcellulose, hydroxypropylcellulose, hydroxypropylmethylcellulose and hydroxyethylcellulose (most preferred, hydroxypropyl methylcellulose). Other cellulose ethers have been described (Alderman, Int. J. Pharm. Tech. & Prod. Mfr. 1984, 5(3) 1-9). Factors affecting drug release are well known to the skilled artisan and have been described in the art (Bamba et al., Int. J. Pharm. 1979, 2, 307).

In other embodiments, enteric-coated preparations can be used for oral sustained release administration. Coating materials include polymers with a pH-dependent solubility (i.e., pH-controlled release), polymers with a slow or pH-dependent rate of swelling, dissolution or erosion (i.e., time-controlled release), polymers that are degraded by enzymes (i.e., enzyme-controlled release) and polymers that form firm layers that are destroyed by an increase in pressure (i.e., pressure-controlled release).

In other embodiments, osmotic delivery systems are used for oral sustained release administration (Verma et al., Drug Dev. Ind. Pharm., 2000, 26:695-708). In some embodiments, OROS™ osmotic devices are used for oral sustained release delivery devices (Theeuwes et al., U.S. Pat. No. 3,845,770; Theeuwes et al., U.S. Pat. No. 3,916,899).

In yet other embodiments, a controlled-release system can be placed in proximity of the target of ABDNAZ and/or pharmaceutical composition, thus requiring only a fraction of the systemic dose (e.g., Goodson, in “Medical Applications of Controlled Release,” supra, vol. 2, pp. 115-138 (1984)). Other controlled-release systems previously may also be used (Langer, 1990, Science 249:1527-1533).

In other embodiments, the particles are dispersed in a dedusting additive. For example, the dedusting agent is polyethylene glycol, e.g., PEG-400.

In another aspect, the present invention provides a mixture comprising a composition or a pharmaceutical composition disclosed herein and a blood sample.

In some embodiments, the concentration of the compound of Formula I is from 0.1 mg/mL of blood to 10 mg/mL of blood, or 0.2 mg/mL of blood to 5 mg/mL of blood, or 0.4 mg/mL of blood to 2.5 mg/mL of blood. Depending upon the circumstances, it may be helpful to administer the compound of Formula I at a concentration of 1 mg of compound per 2.5 mL of blood.

### Dose of ABDNAZ Administered

Exemplary dosing amounts of ABDNAZ are provided according to the number of milligrams of ABDNAZ to be administered to the patient based on the surface area of the patient as measured in m². In certain embodiments, the dose ABDNAZ administered to the patient is from about 1 mg/m² to about 2 mg/m², about 2 mg/m² to about 4 mg/m², about 4 mg/m² to about 6 mg/m², about 6 mg/m² to about 8 mg/m², about 8 mg/m² to about 10 mg/m², about 10 mg/m² to about 12 mg/m², about 12 mg/m² to about 14 mg/m², about 14 mg/m² to about 16 mg/m², about 16 mg/m² to about 18 mg/m², about 18 mg/m² to about 20 mg/m², about 20 mg/m² to about 25 mg/m², about 25 mg/m² to about 30 mg/m², about 30 mg/m² to about 35 mg/m², about 35 mg/m² to about 40 mg/m², about 40 mg/m² to about 45 mg/m², about 45 mg/m² to about 50 mg/m², about 50 mg/m² to about 60 mg/m², or about 60 mg/m² to about 75 mg/m².

The dose of ABDNAZ administered to the patient may be further characterized according to both the amount of ABDNAZ and the mode of delivery, such as intravenous infusion. Accordingly, in certain embodiments, each dose of the formulation comprising ABDNAZ is administered to the patient by intravenous infusion providing ABDNAZ in an amount ranging from about 1 mg/m² to about 90 mg/m². In certain embodiments, each dose of the formulation comprising ABDNAZ is administered to the patient by intravenous infusion providing ABDNAZ in an amount ranging from about 1 mg/m² to about 10 mg/m². In certain embodiments, each dose of the formulation comprising ABDNAZ is administered to the patient by intravenous infusion providing ABDNAZ in an amount ranging from about 1 mg/m² to about 2.5 mg/m². In certain embodiments, each dose of the formulation comprising ABDNAZ is administered to the patient by intravenous infusion providing ABDNAZ in an amount ranging from about 2.5 mg/m² to about 5 mg/m². In certain embodiments, each dose of the formulation comprising ABDNAZ is administered to the patient by intravenous infusion providing ABDNAZ in an amount ranging from about 5 mg/m² to about 10 mg/m². In certain embodiments, each dose of the formulation comprising ABDNAZ is administered to the patient by intravenous infusion providing ABDNAZ in an amount ranging from about 5 mg/m² to about 7 mg/m². In certain embodiments, each dose of the formulation comprising ABDNAZ is administered to the patient by intravenous infusion providing ABDNAZ in an amount ranging from about 8 mg/m² to about 9 mg/m². In certain embodiments, each dose of the formulation comprising ABDNAZ is administered to the patient by intravenous infusion providing ABDNAZ in an amount ranging from about 10 mg/m² to about 20 mg/m². In certain embodiments, each dose of the formulation comprising ABDNAZ is administered to the patient by intravenous infusion providing ABDNAZ in an amount ranging from about 1 mg/m² to about 1.5 mg/m², about 1.5 mg/m² to about 2 mg/m², about 2 mg/m² to about 2.5 mg/m², about 2.5 mg/m² to about 3 mg/m², about 3 mg/m² to about 3.5 mg/m², about 3.5 mg/m² to about 4 mg/m², about 4 mg/m² to about 4.5 mg/m², about 4.5 mg/m² to about 5 mg/m², about 5 mg/m² to about 5.5 mg/m², about 5.5 mg/m² to about 6 mg/m², about 6 mg/m² to about 6.5 mg/m², about 6.5 mg/m² to about 7 mg/m², about 7 mg/m² to about 7.5 mg/m², about 7.5 mg/m² to about 8 mg/m², about 8 mg/m² to about 8.5 mg/m², about 8.5 mg/m² to about 9 mg/m², about 9 mg/m² to about 9.5 mg/m², about 9.5 mg/m² to about 10 mg/m², about 10 mg/m² to about 12 mg/m², about 12 mg/m² to about 14 mg/m², about 14 mg/m² to about 16 mg/m², about 16 mg/m² to about 18 mg/m², about 18 mg/m² to about 20 mg/m², about 20 mg/m² to about 25 mg/m², about 25 mg/m² to about 30 mg/m², about 30 mg/m² to about 35 mg/m², about 35 mg/m² to about 40 mg/m², about 40 mg/m² to about 45 mg/m², or about 45 mg/m² to about 50 mg/m². In certain embodiments, each dose of the formulation comprising ABDNAZ is administered to the patient by intravenous infusion providing ABDNAZ in an amount ranging from about 3 mg/m² to about 8 mg/m².

In more specific embodiments, each dose of the compositions disclosed herein is administered to the patient by intravenous infusion providing ABDNAZ in an amount of about 1.25 mg/m². In certain embodiments, each dose of the compositions disclosed herein is administered to the patient by intravenous infusion providing ABDNAZ in an amount of about 2.5 mg/m². In certain embodiments, each dose of the compositions disclosed herein is administered to the patient by intravenous infusion providing ABDNAZ in an amount of about 5 mg/m². In certain embodiments, each dose of the compositions disclosed herein is administered to the patient by intravenous infusion providing ABDNAZ in an amount of about 8.4 mg/m². In certain embodiments, each dose of the compositions disclosed herein is administered to the patient by intravenous infusion providing ABDNAZ in an amount of about 1 mg/m², about 1.5 mg/m², about 2 mg/m², about 2.5 mg/m², about 3 mg/m², about 3.5 mg/m², about 4 mg/m², about 4.5 mg/m², about 5 mg/m², about 5.5 mg/m², about 6 mg/m², about 6.5 mg/m², about 7 mg/m², about 7.5 mg/m², about 8 mg/m², about 8.5 mg/m², about 9 mg/m², about 9.5 mg/m², about 10 mg/m², about 12 mg/m², about 14 mg/m², about 16 mg/m², about 18 mg/m², about 20 mg/m², about 25 mg/m², about 30 mg/m², about 35 mg/m², about 40 mg/m², about 45 mg/m², or about 50 mg/m².

The method described herein may be further a characterized according to the dose of ABDNAZ administered to the patient. The dose of ABDNAZ described herein for use in combination with temozolomide and the radiation therapy has been selected in view of the dosing schedule and amount of temozolomide and the radiation therapy. Dosing amounts of ABDNAZ are provided according to the number of milligrams of ABDNAZ to be administered to the patient based on the surface area of the patient as measured in m².

In certain embodiments, each dose of the compositions disclosed herein is administered to the patient by intravenous infusion. In certain embodiments, each dose of the compositions disclosed herein is administered to the patient by intravenous infusion providing ABDNAZ in an amount ranging from about 2 mg/m² to about 20 mg/m². In certain embodiments, each dose of the compositions disclosed herein is administered to the patient by intravenous infusion providing ABDNAZ in an amount ranging from about 2.5 mg/m² to about 5 mg/m². In certain embodiments, each dose of the compositions disclosed herein is administered to the patient by intravenous infusion providing ABDNAZ in an amount ranging from about 5 mg/m² to about 10 mg/m². In certain embodiments, each dose of the compositions disclosed herein is administered to the patient by intravenous infusion providing ABDNAZ in an amount ranging from about 10 mg/m² to about 16.5 mg/m². In certain embodiments, each dose of the compositions disclosed herein is administered to the patient by intravenous infusion providing ABDNAZ in an amount of about 2.5 mg/m². In certain embodiments, each dose of the compositions disclosed herein is administered to the patient by intravenous infusion providing ABDNAZ in an amount of about 5 mg/m². In certain embodiments, each dose of the compositions disclosed herein is administered to the patient by intravenous infusion providing ABDNAZ in an amount of about 10 mg/m. In certain embodiments, each dose of the compositions disclosed herein is administered to the patient by intravenous infusion providing ABDNAZ in an amount of about 16.5 mg/m².

In certain embodiments, each dose of the compositions disclosed herein is administered to the patient by intravenous infusion providing ABDNAZ in an amount of from about 0.1 mg to about 20 mg. In certain embodiments, each dose of the compositions disclosed herein is administered to the patient by intravenous infusion providing ABDNAZ in an amount of from about 0.1 mg to about 10 mg. In certain embodiments, each dose of the compositions disclosed herein is administered to the patient by intravenous infusion providing ABDNAZ in an amount of from about 0.5 mg to about 4.0 mg. In certain embodiments, each dose of the compositions disclosed herein is administered to the patient by intravenous infusion providing ABDNAZ in an amount of about 0.1 mg, 0.2 mg, 0.3 mg, 0.4 mg, 0.5 mg, 1.0 mg, 1.5 mg, 2.0 mg, 2.5 mg, 3.0 mg, 3.5 mg, 4.0 mg, 4.5 mg, 5.0 mg, 7.5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg or 250 mg. A dose of 0.5 mg to 66 mg of ABDNAZ may be administered to a subject, but the actual dose will be determined based on the various factors disclosed herein.

In one approach for administering ABDNAZ to a subject as described in the following section, the compound is provided as a liquid formulation containing PEG, for example, PEG-400, at a concentration of 2 mg/mL. In one approach, for each 1 mg of the compound in liquid formulation to be administered the compound is combined with 2.5 mL of blood from the subject to produce a mixture, and then the mixture is administered to the subject. For example, for a dose of 0.5 mg (in 0.25 mL of liquid formulation) is combined with 1.25 mL of blood, or for a dose of 66 mg ABDNAZ (in 33 mL of liquid formulation) is combined with 165 mL of blood prior to administration to a subject.

In some embodiments, the amount of the crystalline form of ABDNAZ and/or pharmaceutical composition thereof administered will, of course, be dependent on, among other factors, the subject being treated, the weight of the subject, the severity of the affliction, the manner of administration and the judgment of the prescribing physician. For example, the dosage of ABDNAZ as a crystal and/or pharmaceutical formulation may be delivered by a single administration, by multiple applications or controlled release. In some embodiments, dosing may be repeated intermittently, may be provided alone or in combination with other drugs and may continue as long as required for effective treatment of the disease state or disorder.

Suitable dosage ranges for oral administration are dependent on the efficiency of radiosensitization, but are generally about 0.001 mg to about 100 mg of the crystalline form of ABDNAZ per kg body weight. Dosage ranges may be readily determined by methods known to the artisan of ordinary skill.

Suitable dosage ranges for intravenous (i.v.) administration are about 0.01 mg to about 100 mg per kg/body weight. Suitable dosage ranges for intranasal administration are generally about 0.01 mg/kg body weight to about 1 mg/kg body weight. Suppositories generally contain about 0.01 milligram to about 50 milligrams of ABDNAZ per kg/body weight or comprise ABDNAZ in the range of about 0.5% to about 10% by weight. Recommended dosages for intradermal, intramuscular, intraperitoneal, subcutaneous, epidural, sublingual or intracerebral administration are in the range of about 0.001 mg to about 200 mg per kg/body weight. Effective doses may be extrapolated from dose-response curves derived from in vitro or animal model test systems. Such animal models and systems are well-known in the art.

### Exemplary ABDNAZ Formulations

The present invention provides formulations comprising a crystalline form of ABDNAZ, e.g., formulations containing whole blood (e.g., autologous blood from the patient being treated), ABDNAZ (e.g., a crystalline form of ABDNAZ), an anticoagulant, and optionally one or more of water, a polyethylene glycol, and N,N-dimethylacetamide. In certain embodiments, the ABDNAZ formulation consists essentially of whole blood, ABDNAZ (e.g., a crystalline form of ABDNAZ), and an anticoagulant. In certain embodiments, the ABDNAZ formulation consists of whole blood, ABDNAZ (e.g., a crystalline form of ABDNAZ), an anticoagulant, and optionally one or more of water, a polyethylene glycol, and N,N-dimethylacetamide. In certain embodiments, the ABDNAZ formulation consists of whole blood, ABDNAZ (e.g., a crystalline form of ABDNAZ), an anticoagulant, and optionally one or more of water, a polyethylene glycol having a number-average molecular weight in the range of about 200 g/mol to about 600 g/mol, and N,N-dimethylacetamide. In certain embodiments, the ABDNAZ formulation consists of whole blood, ABDNAZ (e.g., a crystalline form of ABDNAZ), an anticoagulant, water, a polyethylene glycol having a number-average molecular weight in the range of about 200 g/mol to about 600 g/mol, and N,N-dimethylacetamide. In certain embodiments, the ABDNAZ formulation consists of whole blood, ABDNAZ (e.g., a crystalline form of ABDNAZ), an anticoagulant, and optionally one or more of water, a polyethylene glycol having a number-average molecular weight of about 400 g/mol, and N,N-dimethylacetamide. In certain embodiments, the ABDNAZ formulation consists of whole blood, ABDNAZ (e.g., a crystalline form of ABDNAZ), an anticoagulant, water, a polyethylene glycol having a number-average molecular weight of about 400 g/mol, and N,N-dimethylacetamide.

### Anticoagulant

The formulation of a crystalline form of ABDNAZ may be further characterized according to the identity and/or amount of the anticoagulant. Accordingly, in certain embodiments, the anticoagulant comprises one or more of heparin and a citrate salt. In certain embodiments, the anticoagulant is a solution comprising an alkali metal citrate salt, dextrose, and water. In certain embodiments, the anticoagulant is present in the ABDNAZ formulation in an amount ranging from about 0.1% wt/wt to about 15% w/w. In certain embodiments, the anticoagulant is present in the ABDNAZ formulation in an amount ranging from about 1% wt/wt to about 10% w/w. In certain embodiments, the anticoagulant is present in the ABDNAZ formulation in an amount ranging from about 2% wt/wt to about 8% w/w.

The formulation may be further characterized according to the identity of an anticoagulant in the ABDNAZ formulation as described herein. Accordingly, in certain embodiments, the anticoagulant comprises one or more of heparin and a citrate salt. In certain embodiments, the anticoagulant is a solution comprising an alkali metal citrate salt, dextrose, and water.

### Amount of Whole Blood in the ABDNAZ Formulation

The formulation of a crystalline form of ABDNAZ may be further characterized according to the amount of whole blood in the ABDNAZ formulation. Accordingly, in certain embodiments, the whole blood constitutes at least 30% wt/wt of the ABDNAZ formulation. In certain embodiments, the whole blood constitutes at least 40% wt/wt of the ABDNAZ formulation. In certain embodiments, the whole blood constitutes at least 50% wt/wt of the ABDNAZ formulation. In certain embodiments, the whole blood constitutes at least 60% wt/wt of the ABDNAZ formulation. In certain embodiments, the whole blood constitutes at least 75% wt/wt of the ABDNAZ formulation. In certain embodiments, the whole blood constitutes at least 90% wt/wt of the ABDNAZ formulation. In certain embodiments, the whole blood constitutes from about 60%6 wt/wt to about 99% wt/wt of the ABDNAZ formulation. In certain embodiments, the whole blood constitutes from about 70% wt/wt to about 95% wt/wt of the ABDNAZ formulation. In certain embodiments, the whole blood constitutes from about 75% wt/wt to about 90% wt/wt of the ABDNAZ formulation. In certain embodiments, there is from about 5 mL to about 10 mL of whole blood in the ABDNAZ formulation, from about 10 mL to about 15 mL of whole blood in the ABDNAZ formulation, from about 9 mL to about 11 mL of whole blood in the ABDNAZ formulation, from about 10 mL to about 20 mL of whole blood in the ABDNAZ formulation, from about 20 mL to about 30 mL of whole blood in the ABDNAZ formulation, from about 30 mL to about 50 mL of whole blood in the ABDNAZ formulation, from about 50 mL to about 70 mL of whole blood in the ABDNAZ formulation, or from about 70 mL to about 90 mL of whole blood in the ABDNAZ formulation. In certain embodiments, there is from about 90 mL to about 110 mL of whole blood in the ABDNAZ formulation. In certain embodiments, there is from about 95 mL to about 105 mL of whole blood in the ABDNAZ formulation. In certain embodiments, there is about 100 mL of whole blood in the ABDNAZ formulation.

### Volume of ABDNAZ Formulation Administered to a Subject

The method may be further characterized according to the volume of the composition described herein (e.g., an ABDNAZ formulation) administered to the patient. Accordingly, in certain embodiments, the composition described herein (e.g., an ABDNAZ formulation) has a volume in the range of about 10 mL to about 200 mL. In certain embodiments, the composition described herein (e.g., an ABDNAZ formulation) has a volume in the range of about 10 mL to about 15 mL, about 15 mL to about 20 mL, about 20 mL to about 30 mL, or about 30 mL to about 50 mL. In certain embodiments, the composition described herein (e.g., an ABDNAZ formulation) has a volume in the range of about 50 mL to about 200 mL. In certain embodiments, the composition described herein (e.g., an ABDNAZ formulation) has a volume in the range of about 75 mL to about 150 mL. In certain embodiments, the composition described herein (e.g., an ABDNAZ formulation) has a volume in the range of about 90 mL to about 140 mL. In certain embodiments, the composition described herein (e.g., an ABDNAZ formulation) has a volume in the range of about 100 mL to about 140 mL. In certain embodiments, the composition described herein (e.g., an ABDNAZ formulation) has a volume in the range of about 100 mL to about 120 mL.

One exemplary more specific formulation is an intravenous formulation containing ABDNAZ (e.g., a crystalline form of ABDNAZ as disclosed herein) for intravenous administration to a patient, comprising:
a. whole blood in an amount of at least 60% v/v of the formulation;
b. a polyethylene glycol at a concentration of from about 0.4 μL/mL to about 30 μL/mL in the formulation;
c. N,N-dimethylacetamide at a concentration of from about 0.2 μL/mL to about 15 μL/mL in the formulation;
d. ABDNAZ at a concentration of at least 10 μg/mL in the formulation;
e. water; and
f. an anticoagulant.

Another exemplary more specific formulation is a formulation that consists essentially of:
a. whole blood in an amount of at least 60% v/v of the formulation;
b. a polyethylene glycol at a concentration of from about 0.4 μL/mL to about 30 μL/mL in the formulation;
c. N,N-dimethylacetamide at a concentration of from about 0.2 μL/mL to about 15 μL/mL in the formulation;
d. ABDNAZ at a concentration of at least 10 μg/mL in the formulation;
e. water; and
f. an anticoagulant.

Another exemplary more specific formulation is a formulation that consists of:
a. whole blood in an amount of at least 60% v/v of the formulation;
b. a polyethylene glycol at a concentration of from about 0.4 μL/mL to about 30 μL/mL in the formulation;
c. N,N-dimethylacetamide at a concentration of from about 0.2 μL/mL to about 15 μL/mL in the formulation;
d. ABDNAZ at a concentration of at least 10 μg/mL in the formulation;
e. water; and
f. an anticoagulant.

Another exemplary more specific formulation is an intravenous formulation containing ABDNAZ (e.g., a crystalline form of ABDNAZ as disclosed herein) for intravenous administration to a patient, comprising:
a. a blood product (e.g., an erythrocyte cell, blood plasma, or whole blood) in an amount of at least 30% v/v of the formulation;
b. optionally a polyethylene glycol at a concentration of from about 0.4 μL/mL to about 30 L/mL in the formulation;
c. optionally N,N-dimethylacetamide at a concentration of from about 0.2 μL/mL to about 15 μL/mL in the formulation;
d. ABDNAZ at a concentration of at least 10 μg/mL in the formulation;
e. optionally water; and
f. optionally an anticoagulant.

Another exemplary more specific formulation is an intravenous formulation containing ABDNAZ (e.g., a crystalline form of ABDNAZ as disclosed herein) for intravenous administration to a patient, comprising:
a. whole blood in an amount of at least 30% v/v of the formulation;
b. a polyethylene glycol (e.g., at a concentration of from about 0.4 μL/mL to about 30 μL/mL in the formulation);
c. N,N-dimethylacetamide (e.g., at a concentration of from about 0.2 μL/mL to about 15 μL/mL in the formulation);
d. ABDNAZ at a concentration of at least 10 μg/mL in the formulation;
e. water; and
f. an anticoagulant.

Another exemplary more specific formulation is a formulation that consists essentially of:
a. whole blood in an amount of at least 30% v/v of the formulation;
b. a polyethylene glycol (e.g., at a concentration of from about 0.4 μL/mL to about 30 μL/mL in the formulation);
c. N,N-dimethylacetamide (e.g., at a concentration of from about 0.2 μL/mL to about 15 μL/mL in the formulation);
d. ABDNAZ at a concentration of at least 10 μg/mL in the formulation;
e. water; and
f. an anticoagulant.

### Exemplary Features of Intravenous Formulation

The intravenous formulation may be characterized according to, for example, the identity of a polyethylene glycol, anticoagulant, concentration of ABDNAZ (e.g., a crystalline form of ABDNAZ as disclosed herein), amount of whole blood and other features described herein below.

### Polyethylene Glycol

The formulation may be further characterized according to the identity of a polyethylene glycol in the ABDNAZ formulation as described herein. Accordingly, in certain embodiments, the polyethylene glycol is a polyethylene glycol having a number-average molecular weight in the range of about 200 g/mol to about 600 g/mol. In certain embodiments, the polyethylene glycol is a polyethylene glycol having a number-average molecular weight of about 400 g/mol.

In certain embodiments, the polyethylene glycol is present at a concentration of from about 0.4 μL/mL to about 4 μL/mL in the formulation. In certain embodiments, the N,N-dimethylacetamide at a concentration of from about 0.2 μL/mL to about 2 μL/mL in the formulation.

### Concentration of ABDNAZ

The formulation may be further characterized according to the concentration of ABDNAZ in the ABDNAZ formulation as described herein. Accordingly, in certain embodiments, the ABDNAZ formulation contains ABDNAZ at a concentration of at least 20 μg/mL. In certain embodiments, the ABDNAZ formulation contains ABDNAZ at a concentration of at least 50 μg/mL. In certain embodiments, the ABDNAZ formulation contains ABDNAZ at a concentration of at least 100 μg/mL. In certain embodiments, the ABDNAZ formulation contains ABDNAZ at a concentration of at least 150 μg/mL. In certain embodiments, the ABDNAZ formulation contains ABDNAZ at a concentration in the range of about 10 μg/mL to about 1 mg/mL. In certain embodiments, the ABDNAZ formulation contains ABDNAZ at a concentration in the range of about 10 μg/mL to about 0.5 mg/mL. In certain embodiments, the ABDNAZ formulation contains ABDNAZ at a concentration in the range of about 10 μg/mL to about 250 μg/mL. In certain embodiments, the ABDNAZ formulation contains ABDNAZ at a concentration in the range of about 20 μg/mL to about 200 μg/mL.

### Amount of Whole Blood

The formulation may be further characterized according to the amount of whole blood in the ABDNAZ formulation as described herein. Accordingly, in certain embodiments, the whole blood constitutes at least 30% wt/wt of the formulation. In certain embodiments, the whole blood constitutes at least 40% wt/wt of the formulation. In certain embodiments, the whole blood constitutes at least 50% wt/wt of the formulation. In certain embodiments, the whole blood constitutes at least 75% wt/wt of the formulation. In certain embodiments, the whole blood constitutes at least 90% wt/wt of the formulation. In certain embodiments, the whole blood constitutes from about 60% wt/wt to about 99% wt/wt of the formulation. In certain embodiments, the whole blood constitutes from about 70% wt/wt to about 95% wt/wt of the formulation. In certain embodiments, the whole blood constitutes from about 75% wt/wt to about 90% wt/wt of the formulation. In certain embodiments, there is from about 90 mL to about 110 mL of whole blood in the formulation. In certain embodiments, wherein there is from about 95 mL to about 105 mL of whole blood in the formulation. In certain embodiments, there is about 100 mL of whole blood in the formulation.

### Unit Dose Form of Intravenous Formulation

The formulation may be further characterized according to the volume of a unit dose of the ABDNAZ formulation as described herein. Accordingly, in certain embodiments, the formulation is in the form of a unit dose having a volume in the range of about 10 mL to about 200 mL. In certain embodiments, the formulation is in the form of a unit dose having a volume in the range of about 10 mL to about 15 mL, about 15 mL to about 20 mL, about 20 mL to about 30 mL, about 30 mL to about 40 mL, or about 40 mL to about 50 mL. In certain embodiments, the formulation is in the form of a unit dose having a volume in the range of about 50 mL to about 200 mL. In certain embodiments, the formulation is in the form of a unit dose having a volume in the range of about 75 mL to about 150 mL. In certain embodiments, the formulation is in the form of a unit dose having a volume in the range of about 90 mL to about 140 mL. In certain embodiments, the formulation is in the form of a unit dose having a volume in the range of about 100 mL to about 140 mL. In certain embodiments, the formulation is in the form of a unit dose having a volume in the range of about 100 mL to about 120 mL.

### Characterization of Pain Effect Upon Intravenous Administration to a Subject

The formulation as described herein may be further characterized according to the extent of pain experienced by the patient upon intravenous administration of the ABDNAZ formulation to the patient. Accordingly, in certain embodiments, the formulation is characterized by the feature that any pain experienced by the patient at the site of intravenous administration due to intravenous administration of the formulation to the patient at a rate in the range of 10 mL/hour to 50 mL/hour is no greater than Grade 2. In certain embodiments, wherein the formulation is characterized by the feature that any pain experienced by the patient at the site of intravenous administration due to intravenous administration of the formulation to the patient at a rate in the range of 10 mL/hour to 50 mL/hour is no greater than Grade 1.

The description above describes multiple aspects and embodiments of the invention. The patent application specifically contemplates all combinations and permutations of the aspects and embodiments.

### V. Therapeutic Applications

The present invention provides, in part, methods of using a crystalline form of 2-bromo-1-(3,3-dinitroazetidin-1-yl)ethanone (ABDNAZ) or pharmaceutical compositions thereof to treat or prevent diseases associated with abnormal cell proliferation.

In one aspect, provided are methods of using a crystalline form of ABDNAZ described herein to treat, prevent, or mitigate any of the diseases or conditions described herein. In some aspect, provided are uses of any of the crystalline form of ABDNAZ or composition described herein in the manufacture of a medicament to treat, prevent, or mitigate any of the diseases or conditions described herein. In some aspect, provided are the crystalline form of ABDNAZ and composition described herein for use in the treatment, prevention, or mitigation of any of the diseases or conditions described herein. In some embodiments, the disease is associated with abnormal cell proliferation.

In one aspect, provided are methods of treating or preventing a cancer. In some embodiments, the method comprises administering to a subject in need thereof an effective amount of any of the crystal or composition described herein. In some embodiments, the subject is mammal. In some embodiments, the subject is human. In some embodiments, the administration is performed intravenously or orally.

In another aspect, provided are methods of treating or preventing an ischemic or hypoxic condition. In some embodiments, the method comprises administering to a subject in need thereof an effective amount of any of the crystal or composition described herein. In some embodiments, the subject is mammal. In some embodiments, the subject is human. In some embodiments, the administration is performed intravenously or orally.

In another aspect, provided are methods of treating or preventing neurodegenerative, allergic, autoimmune, fibrotic, inflammatory, infectious, pulmonary, cardiac, vascular, or metabolic diseases. In some embodiments, the method comprises subcutaneously administering to a subject in need thereof an effective amount of any of the crystal or composition described herein. In some embodiments, the subject is mammal. In some embodiments, the subject is human.

In another aspect, provided are methods of protecting against normal tissue toxicity caused by chemotherapy and/or radiation therapy. In some embodiments, the method comprises subcutaneously administering to a subject in need thereof an effective amount of any of the crystal or composition described herein before the subject is exposed to the chemotherapy and/or radiation therapy. In some embodiments, the subject is mammal. In some embodiments, the subject is human.

In another aspect, provided are methods of treating a patient suffering from reduced blood volume or low perfusion. In some embodiments, the method comprises administering to a patient in need thereof a blood product comprising any of the crystal or composition described herein. In some embodiments, the subject is mammal. In some embodiments, the subject is human.

In some embodiments, the methods generally involve administering to a patient in need of such treatment or prevention a therapeutically effective amount of a composition disclosed herein. In certain circumstances, 2-bromo-1-(3,3-dinitroazetidin-1-yl)ethanone (ABDNAZ) is intracellularly activated by the reducing environment of a tumor cell. In other circumstances, the subject is irradiated to activate ABDNAZ. Without wishing to be bound by theory, irradiation or reduction of 2-bromo-1-(3,3-dinitroazetidin-1-yl)ethanone (ABDNAZ) may lead to formation of free radicals that subsequently prevent cell replication and kill cells, presumably by interfering with DNA replication and/or reacting with cell membranes. However, other mechanisms, presently unknown, may account for the efficacy of 2-bromo-1-(3,3-dinitroazetidin-1-yl)ethanone (ABDNAZ) in treating or preventing abnormal cell proliferation. Under other circumstances, 2-bromo-1-(3,3-dinitroazetidin-1-yl)ethanone (ABDNAZ) may be activated by both intracellular reduction and external irradiation. In these embodiments, a synergistic or additive effect may be observed.

In another aspect, the present invention provides a method treating diseases or disorders characterized by abnormal cell proliferation in a subject in need thereof, the method comprising administering to the subject an effective amount of the composition described herein, the pharmaceutical composition described herein or the mixture described herein, thereby to treat the abnormal cell proliferation in the subject.

In some embodiments, the diseases or disorders characterized by abnormal cell proliferation are inflammation, cardiovascular disease and autoimmune disease. In some embodiments, the inflammatory disease is arthritis, diabetic retinopathy, diabetes, rheumatoid arthritis, neovascular glaucoma and psoriasis. In some embodiments, the cardiovascular disease is arteriosclerosis, pulmonary hypertension, systemic hypertension, angina, Cardiac Syndrome X, myocardial infarction, peripheral artery disease, or Raynaud's disease.

In another aspect, the present invention provides a method treating cancer in a subject in need thereof, the method comprising administering to the subject an effective amount of the composition described herein, the pharmaceutical composition described herein or the mixture described herein, thereby to treat the cancer in the subject.

In some embodiments, the cancers are vascularized solid tumor cancers, including but not limited to, carcinomas of the lung, breast, ovary, stomach, pancreas, larynx, esophagus, testes, liver, parotid, biliary tract, colon, rectum, cervix, uterus, endometrium, kidney, bladder, prostrate, thyroid, squamous cell carcinomas, adenocarcinomas, small cell carcinomas, melanomas, gliomas, including, but not limited to, astrocytomas, glioblastomas; neuroblastomas, sarcomas, including, but not limited to, angiosarcomas, chondrosarcomas.

In another aspect, the present invention provides a method treating a hemolytic, condition in a subject in need thereof, the method comprising administering to the subject an effective amount of the composition described herein, the pharmaceutical composition described herein or the mixture described herein, thereby to treat the hemolytic condition in the subject.

In some embodiments, the hemolytic condition is sickle cell disease. In other embodiments, the hemolytic condition is selected from one of the following exemplary hemolytic conditions including sickle cell crisis, thalassemia, hemoglobin C disease, hemoglobin SC disease, sickle thalassemia, hereditary spherocytosis, hereditary elliptocytosis, hereditary ovalcytosis, glucose-6-phosphate deficiency and other red blood cell enzyme deficiencies, paroxysmal nocturnal hemoglobinuria (PNH), paroxysmal cold hemoglobinuria (PCH), thrombotic thrombocytopenic purpura/hemolytic uremic syndrome (TTP/HUS), idiopathic autoimmune hemolytic anemia, drug-induced immune hemolytic anemia, secondary immune hemolytic anemia, non-immunehemolytic anemia caused by chemical or physical agents, malaria, falciparum malaria, bartonellosis, babesiosis, clostridial infection, severe Haemophilus influenzae type b infection, extensive burns, transfusion reaction, rhabdomyolysis (myoglobinemia), transfusion of aged blood, cardiopulomonary bypass, and hemodialysis.

In some embodiments, the composition described herein or the pharmaceutical composition described herein is combined with blood harvested from the subject to create a mixture, whereupon the mixture is administered to the subject. In some embodiments, the blood is whole blood, e.g., autologous or allogeneic whole blood. In other embodiments the blood is a blood product, including but not limited to one or more of plasma, erythrocytes. In another embodiment, the composition described herein or the pharmaceutical composition described herein is combined with blood product for donation to a patient suffering from reduced blood volume or low perfusion, who is suffering from hemorrhagic shock. In another embodiment, the composition described herein or the pharmaceutical composition described herein is administered to the patient separately from the blood product.

In another aspect, the present invention provides a method for in-vitro sterilization. Biological solutions may be treated with the invention, which are toxic to pathogenic bacteria, viruses and cells. This process can also be catalyzed by the application of external energy such as light and heat.

In another aspect, the present invention provides a method of treatment for a patient suffering from a bacterial infection. In some embodiments, the bacterial infection may be a gram-positive bacterial infection or a gram negative bacterial infection. In certain embodiments, the bacterial infection is a gram-positive cocci bacterial infection or a gram-positive bacilli bacterial infection. In certain other embodiments, the bacterial infection is a gram-negative bacterial infection. In certain other embodiments, the bacterial infection is a gram-negative cocci bacterial infection or a gram negative bacilli bacterial infection.

The type of bacterial infection can also be characterized according to whether the bacterial infection is caused by anaerobic or aerobic bacteria. In certain embodiments, the bacterial infection is an anaerobic bacterial infection. In certain other embodiments, the bacterial infection is an aerobic bacterial infection.

In certain embodiments, the bacterial infection is a mycobacterial infection. In more particular embodiments, the bacterial infection is an infection of bacteria selected from the group comprising Mycobacterium tuberculosis, Staphylococcus aureus, Staphylococcus epidermidis, Enterococcus faecalis, Enterococcus faecium, Streptococcus pneumoniae, Streptococcus pyogenes, Mycobacterium smegmatis, Bacillus anthracis, Escherichia coli, Proteus mirabilis, Pseudomonas aeruginosa, Acinetobacter baumannii, Yersinia enterocolitica, Francisella tularensis, Eubacterium lentum, Bacteroides fragilis, Fusobacterium nucleatum, Porphyromonas asaccharolyticus, Clostridium perfringens, and Clostridium difficile. In still other embodiments the bacterial infection is an infection of Mycobacterium tuberculosis bacteria (abbreviated as “MTB” or “TB”).

In certain other embodiments, the bacterial infection is due to a member of the genus Peptostreptococci, a Peptostreptococci asaccharolyticus, a Peptostreptococci magmus, a Peptostreptococci micros, a Peptostreptococci prevotii, a member of the genus Porphyromonas, a Porphyromonas asaccharolytica, a Porphyromonas canoris, a Porphyromonas gingivalis, a Porphyromonas macaccae, a member of the genus Actinomyces, an Actinomyces israelii, an Actinomyces odontolyticus, a member of the genus Clostridium, a Clostridium innocuum, a Clostridium clostridioforme, a Clostridium difficile, a member of the genus Anaerobiospirillum, a member of the genus Bacteroides, a Bacteroides tectum, a Bacteroides ureolyticus, a Bacteroides gracilis (Campylobacter gracilis), a member of the genus Prevotella, a Prevotella intermedia, a Prevotella heparinolytica, a Prevotella orisbuccae, a Prevotella bivia, a Prevotella melaninogenica, a member of the genus Fusobacterium, a Fusobacterium naviforme, a Fusobacterium necrophorum, a Fusobacterium varium, a Fusobacterium ulcerans, a Fusobacterium russii, a member of the genus Bilophila or a Bilophila wadsworthia.

In certain other embodiments, the bacterial infection is due to an antibiotic-resistant bacteria, both aerobic and anaerobic, Gram positive and Gram negative.

In another aspect, the present invention provides a method of treating or preventing an ischemic or hypoxic condition in a subject in need thereof, the method comprising administering to the subject a therapeutically effective amount of the composition describe herein or a pharmaceutical composition described herein or a mixture described herein. In some embodiments, the subject is a mammal.

In some embodiments, the ischemic condition is an acute or chronic ischemic condition. In other embodiments, the acute ischemic condition is myocardial infarction, ischemic stroke, pulmonary embolism, perinatal hypoxia, circulatory shock, mountain sickness or acute respiratory failure. In certain embodiments, the chronic ischemic condition is atherosclerosis, chronic venous insufficiency, chronic heart failure, cardiac cirrhosis, diabetes, macular degeneration, sleep apnea, Raynaud's disease, systemic sclerosis, nonbacterial thrombotic endocarditis, occlusive artery disease, angina pectoris, transient ischemic attacks, or chronic alcoholic liver disease. In some embodiments, the hypoxic condition is cancer, gastric or duodenal ulcers, liver or renal disease, thrombocytopenia, a blood coagulation disorder, a chronic illness, a therapeutic intervention that produces anemia such as cancer chemotherapy or altitude sickness. In other embodiments, the cancer is bladder cancer, breast cancer, clear cell kidney cancer, head/neck squamous cell carcinoma, lung squamous cell carcinoma, malignant melanoma, colorectal cancer, head and neck cancer, cervical cancer, non-small-cell lung cancer (NSCLC), ovarian cancer, pancreatic cancer, prostate cancer, renal cell cancer, small-cell lung cancer (SCLC), triple negative breast cancer, acute lymphoblastic leukemia (ALL), acute myeloid leukemia (AML), chronic lymphocytic leukemia (CLL), chronic myeloid leukemia (CML), diffuse large B-cell lymphoma (DLBCL), EBV-positive DLBCL, primary mediastinal large B-cell lymphoma, T-cell/histiocyte-rich large B-cell lymphoma, follicular lymphoma, Hodgkin's lymphoma (HL), mantle cell lymphoma (MCL), multiple myeloma (MM), myeloid cell leukemia-1 protein (Mcl-1), myelodysplasia syndrome (MDS), non-Hodgkin's lymphoma (NHL), or small lymphocytic lymphoma (SLL).

Additional medical conditions contemplated for treatment or prevention using compositions described herein include nitrogen oxide related rheumatoid arthritis, diabetes (including neuropathies and vasculopathies), and systemic lupus erythematosus.

A contemplated pharmaceutical composition may comprise at least 0.5 mg of the compound of Formula I and is administered intravenously, nasally, optically, intraperitoneally, subcutaneously, or orally.

### Type of Cancer

When a composition disclosed herein is being administered to a subject suffering from cancer in order to treat the cancer, the method may be further characterized according to type of cancer to be treated. For example, in certain embodiments, the cancer is a solid tumor. For example, the cancer can brain cancer, bladder cancer, breast cancer, cervical cancer, cholangiocarcinoma, colon cancer, colorectal cancer, endometrial cancer, esophageal cancer, lung cancer, liver cancer, melanoma, ovarian cancer, pancreatic cancer, prostate cancer, rectal cancer, renal cancer, stomach cancer, testicular cancer, or uterine cancer.

In certain embodiments, the cancer is brain cancer. In certain embodiments, the cancer is colorectal cancer. In certain embodiments, the cancer is cholangiocarcinoma or lung cancer. In certain embodiments, the cancer is lung cancer. In certain embodiments, the lung cancer is small cell lung cancer. In certain other embodiments, the cancer is non-small cell lung cancer. In certain embodiments, the cancer is a leukemia or lymphoma. In certain embodiments, the cancer is a B-cell lymphoma or non-Hodgkin lymphoma.

Additional exemplary cancers for treatment include, for example, bladder cancer, breast cancer, cervical cancer, colon cancer, colorectal cancer, endometrial cancer, esophageal cancer, leukemia, lung cancer, liver cancer, melanoma, ovarian cancer, pancreatic cancer, prostate cancer, rectal cancer, renal cancer, stomach cancer, testicular cancer, and uterine cancer.

It is contemplated that the cancer can be a vascularized tumor, squamous cell carcinoma, adenocarcinoma, small cell carcinoma, melanoma, glioma, neuroblastoma, sarcoma (e.g., an angiosarcoma or chondrosarcoma), larynx cancer, parotid cancer, biliary tract cancer, thyroid cancer, acral lentiginous melanoma, actinic keratoses, acute lymphocytic leukemia, acute myeloid leukemia, adenoid cystic carcinoma, adenomas, adenosarcoma, adenosquamous carcinoma, anal canal cancer, anal cancer, anorectum cancer, astrocytic tumor, bartholin gland carcinoma, basal cell carcinoma, biliary cancer, bone cancer, bone marrow cancer, bronchial cancer, bronchial gland carcinoma, carcinoid, cholangiocarcinoma, chondosarcoma, choriod plexus papilloma/carcinoma, chronic lymphocytic leukemia, chronic myeloid leukemia, clear cell carcinoma, connective tissue cancer, cystadenoma, digestive system cancer, duodenum cancer, endocrine system cancer, endodermal sinus tumor, endometrial hyperplasia, endometrial stromal sarcoma, endometrioid adenocarcinoma, endothelial cell cancer, ependymal cancer, epithelial cell cancer, Ewing's sarcoma, eye and orbit cancer, female genital cancer, focal nodular hyperplasia, gallbladder cancer, gastric antrum cancer, gastric fundus cancer, gastrinoma, glioblastoma, glucagonoma, heart cancer, hemangiblastomas, hemangioendothelioma, hemangiomas, hepatic adenoma, hepatic adenomatosis, hepatobiliary cancer, hepatocellular carcinoma, Hodgkin's disease, ileum cancer, insulinoma, intaepithelial neoplasia, interepithelial squamous cell neoplasia, intrahepatic bile duct cancer, invasive squamous cell carcinoma, jejunum cancer, joint cancer, Kaposi's sarcoma, pelvic cancer, large cell carcinoma, large intestine cancer, leiomyosarcoma, lentigo maligna melanomas, lymphoma, male genital cancer, malignant melanoma, malignant mesothelial tumors, medulloblastoma, medulloepithelioma, meningeal cancer, mesothelial cancer, metastatic carcinoma, mouth cancer, mucoepidermoid carcinoma, multiple myeloma, muscle cancer, nasal tract cancer, nervous system cancer, neuroepithelial adenocarcinoma nodular melanoma, non-epithelial skin cancer, non-Hodgkin's lymphoma, oat cell carcinoma, oligodendroglial cancer, oral cavity cancer, osteosarcoma, papillary serous adenocarcinoma, penile cancer, pharynx cancer, pituitary tumors, plasmacytoma, pseudosarcoma, pulmonary blastoma, rectal cancer, renal cell carcinoma, respiratory system cancer, retinoblastoma, rhabdomyosarcoma, sarcoma, serous carcinoma, sinus cancer, skin cancer, small cell carcinoma, small intestine cancer, smooth muscle cancer, soft tissue cancer, somatostatin-secreting tumor, spine cancer, squamous cell carcinoma, striated muscle cancer, submesothelial cancer, superficial spreading melanoma, T cell leukemia, tongue cancer, undifferentiated carcinoma, ureter cancer, urethra cancer, urinary bladder cancer, urinary system cancer, uterine cervix cancer, uterine corpus cancer, uveal melanoma, vaginal cancer, verrucous carcinoma, VIPoma, vulva cancer, well differentiated carcinoma, or Wilms tumor.

The invention also provides therapeutic methods for treating brain metastases. For example, the methods may use a particular dosing regimen of ABDNAZ, radiation therapy, and optionally an additional anti-cancer agent. The therapeutic method can be further characterized according to type of brain metastasis to be treated. For example, the brain metastasis can be characterized according to the type of primary tumor from which the brain metastasis results. In certain embodiments, the brain metastasis is a brain metastasis from a melanoma, lung cancer, breast cancer, colon cancer, kidney cancer, liver cancer, ovarian cancer, pancreatic cancer, prostate cancer, rectal cancer, stomach cancer, testicular cancer, uterine cancer, endometrial cancer, or esophageal cancer. In certain other embodiments, the brain metastasis is a brain metastasis from a melanoma, lung cancer, breast cancer, colon cancer, or kidney cancer. In yet other embodiments, the brain metastasis is from a melanoma.

Without limitation, exemplary cancers from which a brain metastasis may result include, for example, bladder cancer, breast cancer, cervical cancer, colon cancer, colorectal cancer, endometrial cancer, esophageal cancer, leukemia, lung cancer, liver cancer, melanoma, ovarian cancer, pancreatic cancer, prostate cancer, rectal cancer, renal cancer, stomach cancer, testicular cancer, and uterine cancer. In yet other embodiments, the cancer is a vascularized tumor, squamous cell carcinoma, adenocarcinoma, small cell carcinoma, melanoma, glioma, neuroblastoma, sarcoma (e.g., an angiosarcoma or chondrosarcoma), larynx cancer, parotid cancer, biliary tract cancer, thyroid cancer, acral lentiginous melanoma, actinic keratoses, acute lymphocytic leukemia, acute myeloid leukemia, adenoid cystic carcinoma, adenomas, adenosarcoma, adenosquamous carcinoma, anal canal cancer, anal cancer, anorectum cancer, astrocytic tumor, bartholin gland carcinoma, basal cell carcinoma, biliary cancer, bone cancer, bone marrow cancer, bronchial cancer, bronchial gland carcinoma, carcinoid, cholangiocarcinoma, chondosarcoma, choriod plexus papilloma/carcinoma, chronic lymphocytic leukemia, chronic myeloid leukemia, clear cell carcinoma, connective tissue cancer, cystadenoma, digestive system cancer, duodenum cancer, endocrine system cancer, endodermal sinus tumor, endometrial hyperplasia, endometrial stromal sarcoma, endometrioid adenocarcinoma, endothelial cell cancer, ependymal cancer, epithelial cell cancer, Ewing's sarcoma, eye and orbit cancer, female genital cancer, focal nodular hyperplasia, gallbladder cancer, gastric antrum cancer, gastric fundus cancer, gastrinoma, glioblastoma, glucagonoma, heart cancer, hemangiblastomas, hemangioendothelioma, hemangiomas, hepatic adenoma, hepatic adenomatosis, hepatobiliary cancer, hepatocellular carcinoma, Hodgkin's disease, ileum cancer, insulinoma, intaepithelial neoplasia, interepithelial squamous cell neoplasia, intrahepatic bile duct cancer, invasive squamous cell carcinoma, jejunum cancer, joint cancer, Kaposi's sarcoma, pelvic cancer, large cell carcinoma, large intestine cancer, leiomyosarcoma, lentigo maligna melanomas, lymphoma, male genital cancer, malignant melanoma, malignant mesothelial tumors, medulloblastoma, medulloepithelioma, meningeal cancer, mesothelial cancer, metastatic carcinoma, mouth cancer, mucoepidermoid carcinoma, multiple myeloma, muscle cancer, nasal tract cancer, nervous system cancer, neuroepithelial adenocarcinoma nodular melanoma, non-epithelial skin cancer, non-Hodgkin's lymphoma, oat cell carcinoma, oligodendroglial cancer, oral cavity cancer, osteosarcoma, papillary serous adenocarcinoma, penile cancer, pharynx cancer, pituitary tumors, plasmacytoma, pseudosarcoma, pulmonary blastoma, rectal cancer, renal cell carcinoma, respiratory system cancer, retinoblastoma, rhabdomyosarcoma, sarcoma, serous carcinoma, sinus cancer, skin cancer, small cell carcinoma, small intestine cancer, smooth muscle cancer, soft tissue cancer, somatostatin-secreting tumor, spine cancer, squamous cell carcinoma, striated muscle cancer, submesothelial cancer, superficial spreading melanoma, T cell leukemia, tongue cancer, undifferentiated carcinoma, ureter cancer, urethra cancer, urinary bladder cancer, urinary system cancer, uterine cervix cancer, uterine corpus cancer, uveal melanoma, vaginal cancer, verrucous carcinoma, VIPoma, vulva cancer, well differentiated carcinoma, or Wilms tumor.

Combinations with Other Additional Anti-Cancer Agents

In certain embodiments, the method described herein further comprises administering an additional anti-cancer agent to the subject. In certain embodiments, the additional anti-cancer agent is temozolomide, cisplatin, carboplatin, trastuzumab, or sunitinib. In yet other embodiments, the additional anti-cancer agent is temozolomide. In certain embodiments, for any day in which temozolomide is administered to the patient, the temozolomide is administered orally at a dosage of from about 75 mg/m² to about 150 mg/m².

Further exemplary additional anti-cancer agents include, for example, azacitidine, azathioprine, bleomycin, capecitabine, carmustine, chlorambucil, cyclophosphamide, cytarabine, dacarbazine, daunorubicin, docetaxel, doxifluridine, doxorubicin, epirubicin, epothilone, etoposide, fluorouracil, fulvestrant, gemcitabine, hydroxyurea, idarubicin, imatinib, lomustine, mechlorethamine, mercaptopurine, methotrexate, mitoxantrone, oxaliplatin, paclitaxel, pemetrexed, procarbazine, raloxifene, teniposide, thiotepa, tioguanine, tamoxifen, toremifene, valrubicin, vinblastine, vincristine, vindesine, vinorelbine, and pharmaceutically acceptable salts thereof.

In yet other embodiments, the additional anti-cancer agent is abraxane; acivicin; aclarubicin; acodazole hydrochloride; acronine; adozelesin; aldesleukin; altretamine; ambomycin; ametantrone acetate; amrubicin; amsacrine; anastrozole; anthramycin; asparaginase; asperlin; azacitidine; azetepa; azotomycin; batimastat; benzodepa; bicalutamide; bisantrene hydrochloride; bisnafide dimesylate: bizelesin; bleomycin sulfate; brequinar sodium; bropirimine; busulfan; cactinomycin; calusterone; caracemide; carbetimer; carboplatin; carmustine; carubicin hydrochloride; carzelesin; cedefmgol: celecoxib; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; de/.aguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatm; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; herceptin; hydroxyurea; idarubicin hydrochloride; ifosfamide; ilmofosine; iproplatin; irinotecan; irinotecan hydrochloride; lanreotide acetate; lapatinib; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; portiromycin; prednimustine; procarbazine hydrochloride; puromycin; puromycin hydrochloride; pyrazofurin; riboprine; romidepsin; safingol; safingol hydrochloride; semustine; simtrazene; sparfosate sodium; sparsomycin; spirogermanium hydrochloride; spiromustine; spiroplatin; a stem cell treatment; streptonigrin; streptozocin; sulofenur; talisomycin; tecogalan sodium; taxotere; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; or zorubicin hydrochloride.

### Characterization of Anti-Cancer Effects

When a composition described herein is being administered to a cancer patient in order to treat the cancer, the therapeutic method may be further characterized according to the anti-cancer effect of the treatment, such as (i) a reduction in the size of at least one tumor in the patient, and/or (ii) reduction in the number of tumors in the patient.

Accordingly, in certain embodiments, the therapeutic method described herein is characterized by at least a 20% reduction in the size of at least one tumor in the patient. In certain other embodiments, there is at least a 35% reduction in the size of at least one tumor in the patient. In certain other embodiments, there is at least a 50% reduction in the size of at least one tumor in the patient. In certain other embodiments, there is at least a 60%, 70%, 80% or 90% reduction in the size of at least one tumor in the patient. In certain other embodiments, there is about a 5%-50%, 10%-50%, 20%-50%, 5%-75%, 10%-75%, 20%-75%, or 50%-90% reduction in the size of at least one tumor in the patient.

When the cancer to be treated are brain metastases, the method may be further characterized according to the reduction in number and/or size of the brain metastases. In certain embodiments, there is at least a 20% reduction in the number of brain metastases in the patient. In certain other embodiments, there is at least a 35% reduction in the number of brain metastases in the patient. In yet other embodiments, there is at least a 50% reduction in the number of brain metastases in the patient. In certain other embodiments, there is at least a 60%, 70%, 80% or 90% reduction in the number of brain metastases in the patient. In certain other embodiments, there is about a 5%-50%, 10%-50%, 20%-50%, 5%-75%, 10%-75%, 20%-75%, or 50%-90% reduction in the number of brain metastases in the patient.

### Patients for Treatment

The therapeutic method may be further characterized according to the patient to be treated. In certain embodiments, the patient is an adult human. In certain other embodiments, the patient is a pediatric human.

In certain embodiments, the patient does not suffer from anemia or have reduced blood volume. In certain embodiments, the patient has at least 95% of the amount of their average daily blood volume.

### Tissue Protection

Under certain circumstances, it is contemplated that the compounds described herein can be used to protect against normal tissue toxicity that may occur when a subject is undergoing chemotherapy and/or radiation. The method comprising: subcutaneously administering to a subject in need thereof an effective amount of the impact- or detonation-insensitive composition, pharmaceutical composition, or mixture that contains the compound of Formula I as described herein before the subject is exposed to the chemotherapy and/or radiation therapy.

Under certain circumstances, the subject has cancer, for example head and neck cancer. Also, it is contemplated that at least about 0.5 mg (for example, 0.5 mg to 4 mg) of the compound of Formula I is administered to the subject, which can be administered, for example, by injection, in single doses or via multiple divided doses injections.

Under certain circumstance, the normal tissue toxicity to be protected against can be acute mucositis (for example, late mucositis) or dysphagia.

### VI. Kits for Use in Medical Applications

The current invention also provides therapeutic kits comprising ABDNAZ (e.g., a crystalline form of ABDNAZ as disclosed herein) and/or pharmaceutical compositions thereof. The therapeutic kits may also contain other compounds (e.g., chemotherapeutic agents, natural products, apoptosis-inducing agents, etc.) or pharmaceutical compositions thereof.

Therapeutic kits may have a single container which contains ABDNAZ (e.g., a crystalline form of ABDNAZ as disclosed herein) and/or pharmaceutical compositions thereof with or without other components (e.g., other compounds or pharmaceutical compositions of these other compounds) or may have distinct container for each component. In some embodiments, therapeutic kits include ABDNAZ (e.g., a crystalline form of ABDNAZ as disclosed herein) and/or a pharmaceutical composition thereof packaged for use in combination with the co-administration of a second compound (preferably, a chemotherapeutic agent, a natural product, an apoptosis-inducing agent, etc.) or a pharmaceutical composition thereof. The components of the kit may be pre-complexed or each component may be in a separate distinct container prior to administration to a patient.

The components of the kit may be provided in one or more liquid solutions, preferably, an aqueous solution, more preferably, a sterile aqueous solution. The components of the kit may also be provided as solids, which may be converted into liquids by addition of suitable solvents, which are preferably provided in another distinct container.

The container of a therapeutic kit may be a vial, test tube, flask, bottle, syringe, or any other means of enclosing a solid or liquid. Usually, when there is more than one component, the kit will contain a second vial or other container, which allows for separate dosing. The kit may also contain another container for a pharmaceutically acceptable liquid.

Preferably, a therapeutic kit will contain apparatus (e.g., one or more needles, syringes, eye droppers, pipette, etc.), which enables administration of the components of the kit.

### ENUMERATED EMBODIMENTS

The following enumerated embodiments are representative of some aspects of the invention.

1. A composition comprising solid crystalline, non-impact sensitive particles comprising the compound of Formula I:

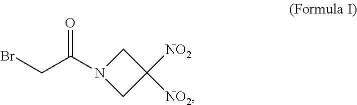 [SMILES: O=C(CBr)N1CC([N+](=O)[O-])([N+](=O)[O-])C1]

or a pharmaceutically acceptable salt thereof, the solid crystalline, non-impact sensitive particles having an angle of repose of less than about 45 degrees.

2. The composition of embodiment 1, wherein the composition comprises the compound in a solvated form.

3. The composition of embodiment 2, wherein the composition comprises tetrahydrofuran (THF).

4. The composition of any one of embodiments 1-3, wherein the composition comprises the particles in a clathrated form.

5. The composition of embodiment 4, wherein the particles comprise THF.

6. The composition of any one of embodiments 1-5, wherein the composition further comprises n-heptane.

7. The composition of any one of embodiments 1-6, wherein the composition has a bulk density in the range of from 0.1 g/cm³ to 0.6 g/cm³.

8. The composition of any one of embodiments 1-7, wherein the bulk density is from 0.15 g/cm³ to 0.5 g/cm³, from 0.15 g/cm³ to 0.4 g/cm³, or from 0.16 g/cm³ to 0.3 mg/cm³.

9. The composition of any one of embodiments 1-8, wherein the particles are dispersed in a dedusting agent.
10. The composition of embodiment 9, wherein the dedusting agent is polyethylene glycol.
11. A pharmaceutical composition comprising the composition of any one of embodiments 1-10, and a pharmaceutically acceptable carrier.

12. The pharmaceutical composition of embodiment 11 further comprising N,N-dimethylacetamide.

13. The pharmaceutical composition of embodiment 11 or 12 further comprising an anti-coagulant.

14. A mixture comprising the composition of any one of embodiments 1-10 or the pharmaceutical composition of any one of embodiments 11-13, and a blood sample.

15. The mixture of embodiment 14, wherein the blood sample has been harvested from a subject to be treated with the compound

16. The mixture of embodiment 15, wherein the concentration of the compound of Formula I is from 0.1 mg/mL of blood to 10 mg/mL of blood.

17. A method of producing a crystalline form of the compound of Formula I, the method comprising the steps of:
(a) dissolving the compound of Formula I in tetrahydrofuran;
(b) adding the solution of step (a) to n-heptane with stirring; and
(c) cooling the solution produced by step (b), thereby to provide a crystalline form of the composition of any one of embodiments 1-8.

18. The method of embodiment 17, wherein the solution produced by step (a) is combined with the heptane in step (b) at a ratio of 1:5 (v/v).

19. The method embodiment 17 or 18, wherein, during step (b), the adding occurs over at least 30 minutes, 45 minutes or one hour.

20. A crystalline form of the compound of Formula I produced by the method of any one of embodiments 17-19.

21. The composition of any one of embodiments 1-10, wherein the composition is not impact sensitive as determined by using a Series 3 Type (a)(ii) Test as set forth in the United Nations Manual of Tests and Criteria, seventh edition, 2019 by exposing a 40 mm³ sample of the composition to 40 J of energy.

22. The crystalline form of the compound of embodiment 20, wherein impact insensitivity of the compound is determined by using a Series 3 Type (a)(ii) Test as set forth in the United Nations Manual of Tests and Criteria, seventh edition, 2019 by exposing a 40 mm³ sample of the compound to 40 J of energy.

23. A method treating cancer in a subject in need thereof, the method comprising administering to the subject an effective amount of the composition of any one of embodiments 1-10, the pharmaceutical composition of any one of embodiments 11-13, or the mixture of any one of embodiments 14-16, thereby to treat the cancer in the subject.

24. The method of embodiment 23, wherein the composition of any one of embodiments 1-10 or 21 or the pharmaceutical composition of any one of embodiments 11-13 is combined with blood harvested from the subject to create a mixture, whereupon the mixture is administered to the subject.

25. A method of treating or preventing an ischemic or hypoxic condition in a subject in need thereof, the method comprising administering to the subject a therapeutically effective amount of the composition of any one of embodiments 1-10 or 21 or a pharmaceutical composition of any one of embodiments 11-13 or a mixture of any one of embodiments 14-16.

26. The method of embodiment 25, wherein the ischemic condition is an acute or chronic ischemic condition.

27. The method of embodiment 26, wherein the acute ischemic condition is myocardial infarction, ischemic stroke, pulmonary embolism, perinatal hypoxia, circulatory shock, mountain sickness or acute respiratory failure.

28. The method of embodiment 26, wherein the chronic ischemic condition is atherosclerosis, chronic venous insufficiency, chronic heart failure, cardiac cirrhosis, diabetes, macular degeneration, sleep apnea, Raynaud's disease, systemic sclerosis, nonbacterial thrombotic endocarditis, occlusive artery disease, angina pectoris, transient ischemic attacks, or chronic alcoholic liver disease.

29. The method of embodiment 25, wherein the hypoxic condition is cancer, gastric or duodenal ulcers, liver or renal disease, thrombocytopenia, a blood coagulation disorder, a chronic illness, a therapeutic intervention that produces anemia such as cancer chemotherapy or altitude sickness.

30. The method of embodiment 29, wherein the cancer is bladder cancer, breast cancer, clear cell kidney cancer, head/neck squamous cell carcinoma, lung squamous cell carcinoma, malignant melanoma, colorectal cancer, head and neck cancer, cervical cancer, non-small-cell lung cancer (NSCLC), ovarian cancer, pancreatic cancer, prostate cancer, renal cell cancer, small-cell lung cancer (SCLC), triple negative breast cancer, acute lymphoblastic leukemia (ALL), acute myeloid leukemia (AML), chronic lymphocytic leukemia (CLL), chronic myeloid leukemia (CML), diffuse large B-cell lymphoma (DLBCL), EBV-positive DLBCL, primary mediastinal large B-cell lymphoma, T-cell/histiocyte-rich large B-cell lymphoma, follicular lymphoma, Hodgkin's lymphoma (HL), mantle cell lymphoma (MCL), multiple myeloma (MM), myeloid cell leukemia-1 protein (Mcl-1), myelodysplasia syndrome (MDS), non-Hodgkin's lymphoma (NHL), or small lymphocytic lymphoma (SLL).

31. The method of any one of embodiments 23-30, wherein the pharmaceutical composition comprises at least 0.5 mg of the compound of Formula I and is administered intravenously, nasally, optically, intraperitoneally, subcutaneously, or orally.

32. A method of protecting against normal tissue toxicity caused by chemotherapy and/or radiation therapy, the method comprising: subcutaneously administering to a subject in need thereof an effective amount of the composition of any one of embodiments 1-10 or 21, the pharmaceutical composition of any one of embodiments 11-13 or the mixture of any one of embodiments 14-16 before the subject is exposed to the chemotherapy and/or radiation therapy.

33. The method of embodiment 32, wherein the subject has cancer.

34. The method of embodiment 33, wherein the cancer is head and neck cancer.

35. The method of any one of embodiments 32-34, wherein at least about 0.5 mg of the compound of Formula I is administered to the subject.

36. The method of embodiment 35, wherein from about 0.5 mg to 4 mg of the compound of Formula I is administered to the subject.

37. The method of embodiment 35 or 36, wherein the amount of the compound of Formula I is administered in one or more divided injections.

38. The method of any one of embodiments 32-37, wherein the normal tissue toxicity is acute mucositis or dysphagia.

39. The method of embodiment 38, wherein the mucositis is late mucositis.

### EXAMPLES

The invention now being generally described, will be more readily understood by reference to the following examples, which are included merely for purposes of illustration of certain aspects and embodiments of the present invention, and are not intended to limit the invention.

### Example 1. Synthesis of an Impact Insensitive Crystalline Form of ABDNAZ

A crystalline form of ABDNAZ was prepared according to the synthetic scheme below.

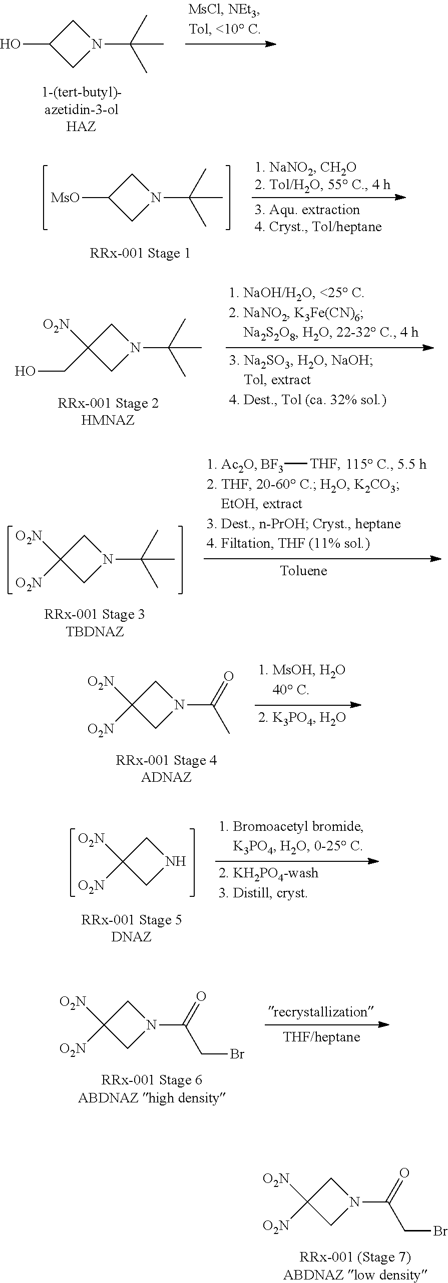

In the final recrystallization step (the step to convert Stage 6 material to Stage 7 material), ABDNAZ was dissolved in THF and the solution of ABDNAZ (5 relative volumes) was added to quickly stirred n-heptane (25 relative volumes) over 1 hour at room temperature. The resulting suspension was cooled to 5° C., stirred for 1 hour, and subsequently isolated by filtration.

The crystalline material when dried and then characterized was analytically pure by LC-purity and assay.

Samples of crystalline material were subjected to a standard drop test pursuant to a Series 3 Type (a)(ii) Test procedure using a BAM Fallhammer as described in United Nations Manual of Tests and Criteria, seventh edition, 2019. Tests were conducted such that 40 mm³ of crystalline material produced by the foregoing method was disposed in an impact device and subjected to 40 J of energy. The test was conducted on six individual samples of crystalline material under the same experimental conditions, and the operator determined whether an explosion occurred. No explosion occurred in any of the samples, and as a result the crystalline material produced in this Example was characterized as not explosive or not impact sensitive. In other words, the material was impact insensitive.

### Example 2. Biological Activity of an Impact Insensitive Crystalline Form of ABDNAZ

Samples 1 and 3 were prepared as impact sensitive crystalline form of ABDNAZ. Samples 2 and 4 were prepared according to the synthetic scheme provided in Example 1 above. Samples 2 and 4 were found to be impact insensitive.

Cancer cell lines HCT-116, SCC VII, and A549 were obtained from the American Type Culture Collection (ATCC, Rockville, MD, USA), and maintained according to ATCC's instructions. All culture reagents were obtained from Invitrogen (Carlsbad, CA, USA).

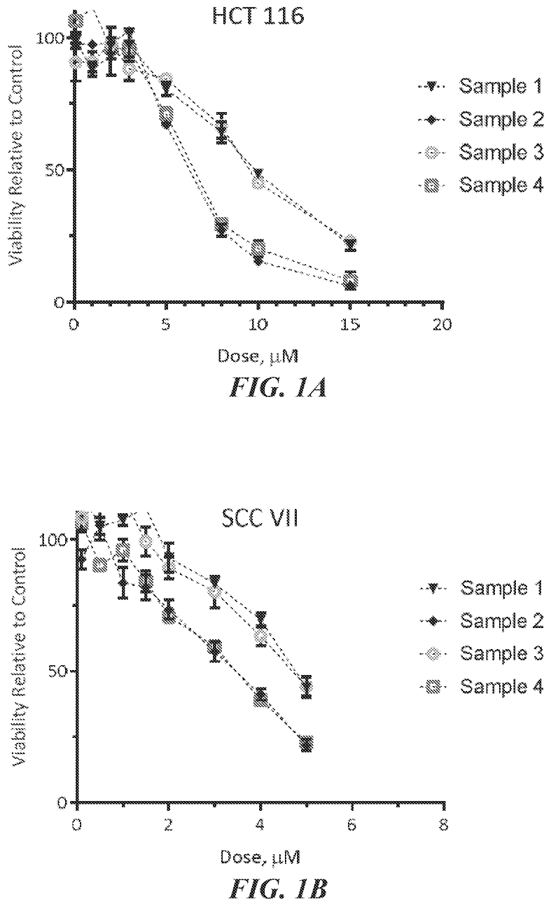

These cancer cell lines were split into 96-well dishes at 2,000 cells per well, and were treated with varying concentrations of sample 1, 2, 3, or 4. Cell proliferation was evaluated using the MTT (3-[4,5-dimethylthiazol-2-yl]-2,5-diphenyl tetrazolium bromide) colorimetric assay 24 hours after treatment with sample 1, 2, 3, or 4. The absorbance of the formazan solution was measured spectrophotometrically at the wavelength of 570 nm. The measured optical density (OD) values were directly proportional to the number of viable cells. Experiments were repeated twice for each combination of cell line, sample, and sample concentration. Percent cell viability relative to the control are plotted in FIGS. 1A to 1C.

The results show that impact insensitive crystalline form of ABDNAZ (samples 2 and 4) is more effective in suppressing viability of cancer cell lines compared to impact sensitive form of ABDNAZ (samples 1 and 3).

### Example 3. Solubility of an Impact Insensitive Crystalline Form of ABDNAZ

Samples 5 and 6 were prepared according to the synthetic scheme provided in Example 1 above. Samples 5 and 6 were found to be impact insensitive. Samples 7 and 8 were prepared as an impact sensitive crystalline form of ABDNAZ.

Solubility of these samples was measured through serial dilution of each sample in DMSO. Assays were performed in 96-well microplates. In each well, 100 μL of DMSO and a set amount of each sample were added. The initial amount of each sample was sufficient to saturate DMSO, which was characterized by depositing of undissolved particles in each well. An incubator shaker was used to keep samples at 25° C. during the test with the agitation of 50 rpm for 4 hours.

Additional DMSO was added to each well to gradually dilute the sample. After each DMSO addition, samples were agitated at 50 rpm for 4 hours, while maintaining the temperature at 25° C. Then, the undissolved amount of each sample was quantified based on the absorption relative to the buffer at 250 nm using a UV-Vis spectrophotometer.

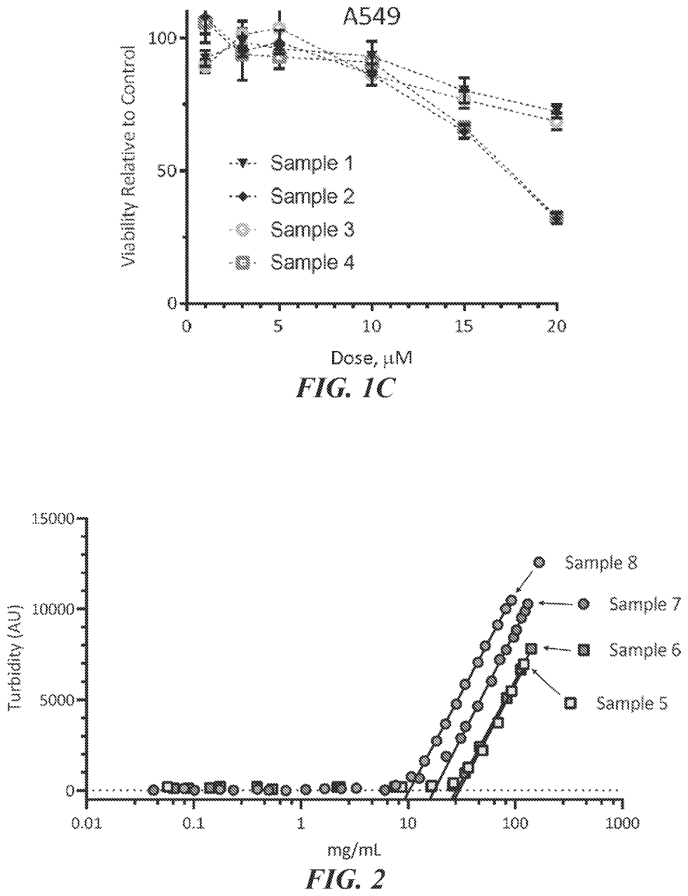

The resulting turbidity is plotted in FIG. 2. Based on this plot, solubility of each sample is determined as summarized below in Table 1.

TABLE 1

| Sample | Solubility (mg/mL) |
|---|---|
| Sample 5 | 28 |
| Sample 6 | 27 |
| Sample 7 | 16 |
| Sample 8 | 12 |

### Example 4. Statistical Analysis of Bulk Density and Size Distribution of RRx-001 Particles

The differences between THF-containing and THF-free samples based on bulk density, D10, D50, and D90 measurements were statistically significant (nonparametric test (Wilcoxon) and Kolmogorov-Smirnov test).

### Bulk Density Measurements Distributional Comparison

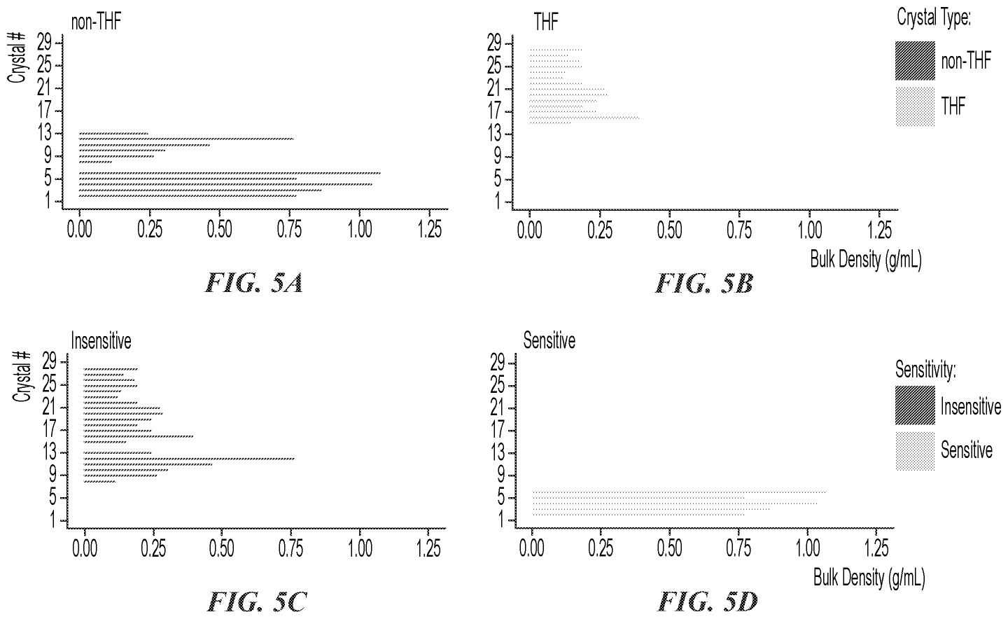

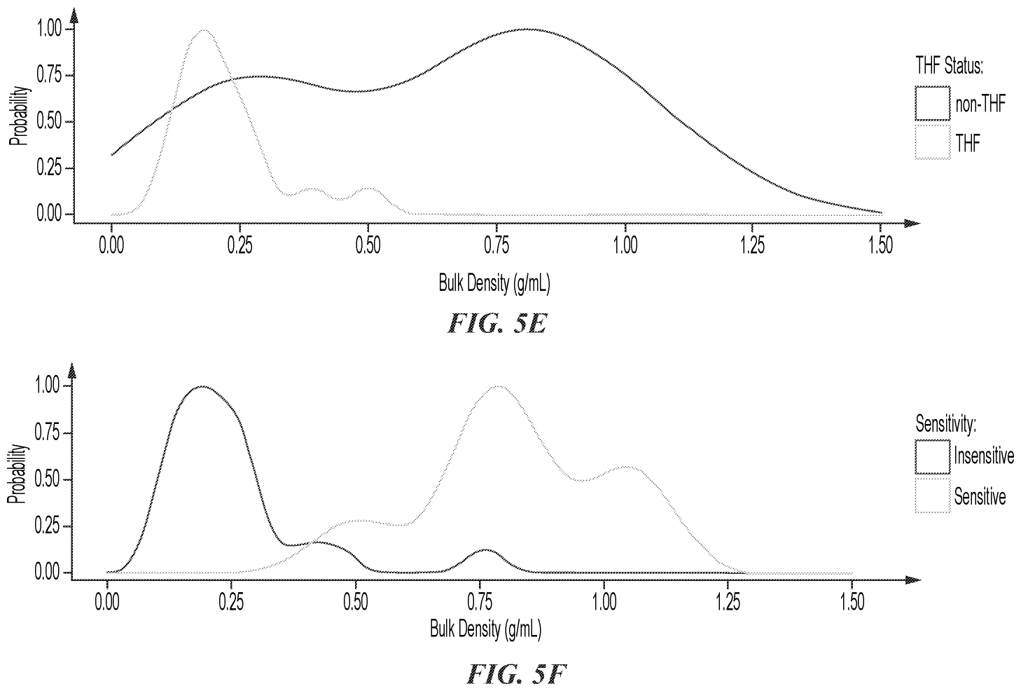

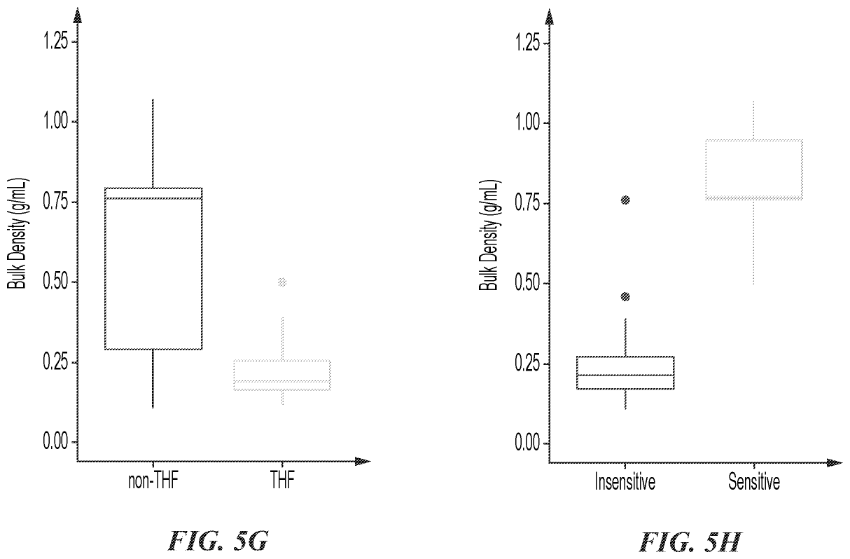

A graphical depiction of the bulk density of crystals reveals an evident separation between THF-containing and THF-free samples, as well as between impact sensitive and impact insensitive samples. FIG. 5A depicts bulk density of RRx-001 particles crystalized without THF; FIG. 5B depicts bulk density of RRx-001 particles crystalized with THF; FIG. 5C depicts bulk density of impact insensitive RRx-001 particles; and FIG. 5D depicts bulk density of impact sensitive RRx-001 particles.

The bulk density empirical probability functions are provided in FIGS. 5E and 5F, and the boxplots are provided in FIGS. 5G and 5H. The visual separation is accompanied by a statistically significant findings based on the nonparametric Wilcoxon rank sum test with continuity correction applied to test the null hypothesis that the true bulk density location shift is zero (i.e., equality of medians) against the alternative hypothesis that the true location shift is not zero. The Wilcoxon test identified a statistically significant difference between impact sensitive and insensitive particles (W statistic=1.5 and a corresponding two-sided p-value=0.0001) and also between THF-containing and THF-free particles (W statistic=153, p-value=0.0022). A Kolmogorov-Smirnov (KS) test comparing the distributions of the bulk density also identified a statistically significant difference between impact sensitive and insensitive particles (KS W statistic=0.95, p=0.0001) and between THF-containing and THF-free particles (KS W statistic=0.6166 and p=0.0125).

### D10 Measurements Distributional Comparison

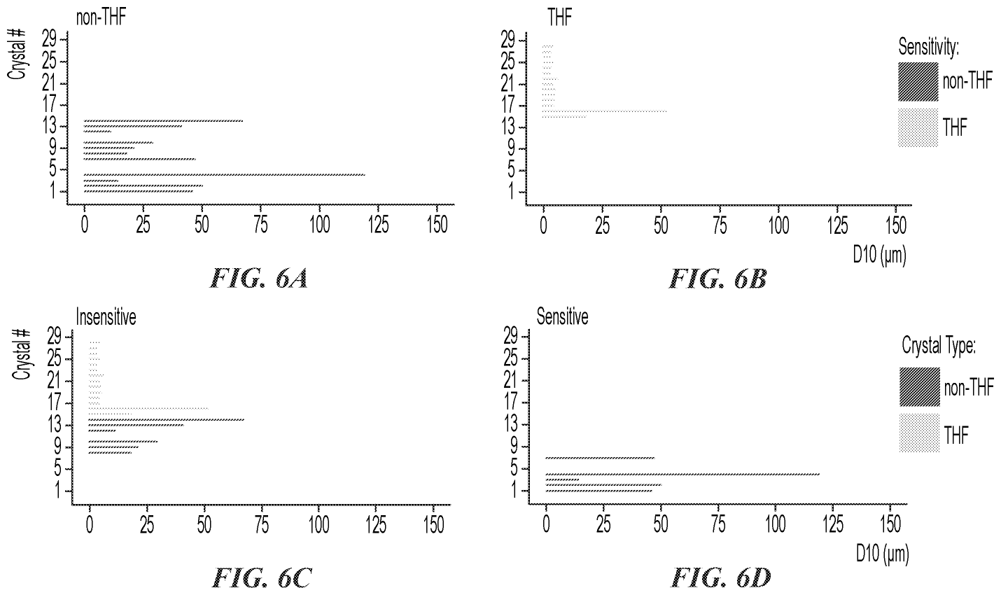

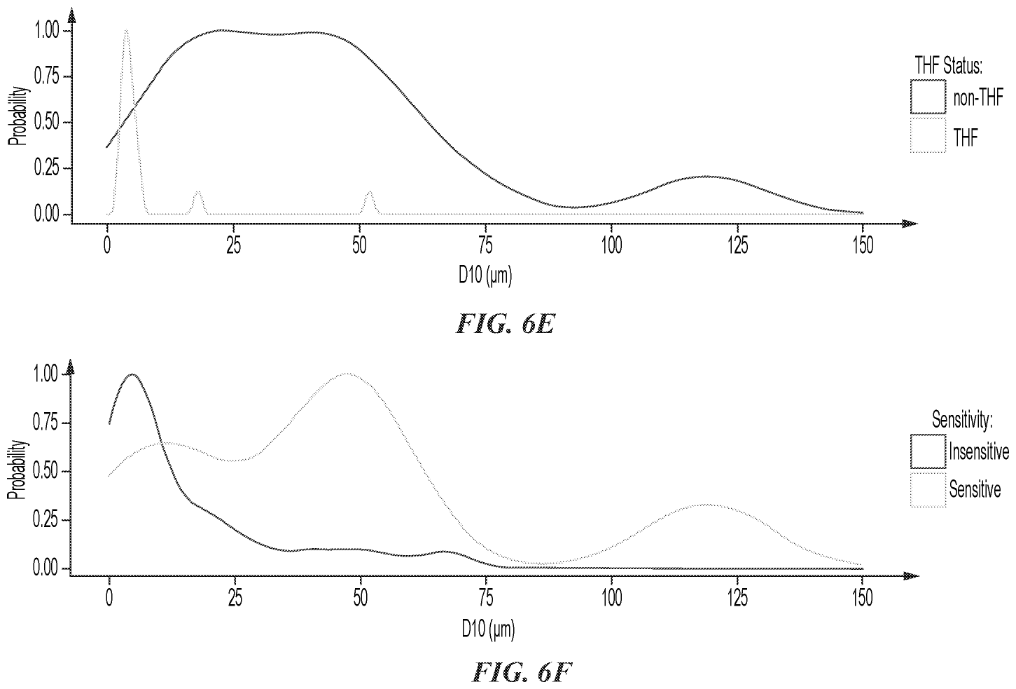

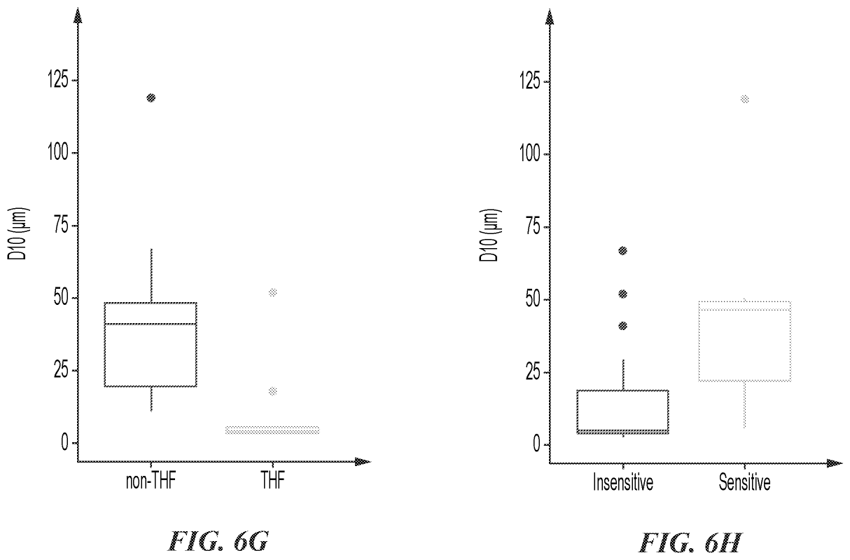

A graphical depiction of D10 measurements reveals an evident separation between THF-containing and THF free samples, as well as between impact sensitive and impact insensitive samples. FIG. 6A depicts D10 of RRx-001 particles crystalized without THF; FIG. 6B depicts D10 of RRx-001 particles crystalized with THF; FIG. 6C depicts D10 of impact insensitive RRx-001 particles; and FIG. 6D depicts D10 of impact sensitive RRx-001 particles.

The D10 empirical probability functions are provided in FIGS. 6E and 6F, and the boxplots are provided in FIGS. 6G and 6H. The visual separation is accompanied by a statistically significant findings based on the nonparametric Wilcoxon rank sum test with continuity correction applied to test the null hypothesis that the true D10 location shift is zero (i.e., equality of medians) against the alternative hypothesis that the true location shift is not zero. The Wilcoxon test identified a statistically significant difference between impact sensitive and insensitive particles (W statistic=21.5 and a corresponding two-sided p-value=0.0200) and also between THF-containing and THF-free particles (W statistic=W statistic=153.5 and a p-value=0.0002). A Kolmogorov-Smirnov (KS) test comparing the distributions of the D10 also identified a statistically significant difference between impact sensitive and insensitive particles (KS W statistic=0.5666, p=0.1032) and between THF-containing and THF-free particles (KS W statistic=0.8666 and p=0.0001).

### D50 Measurements Distributional Comparison

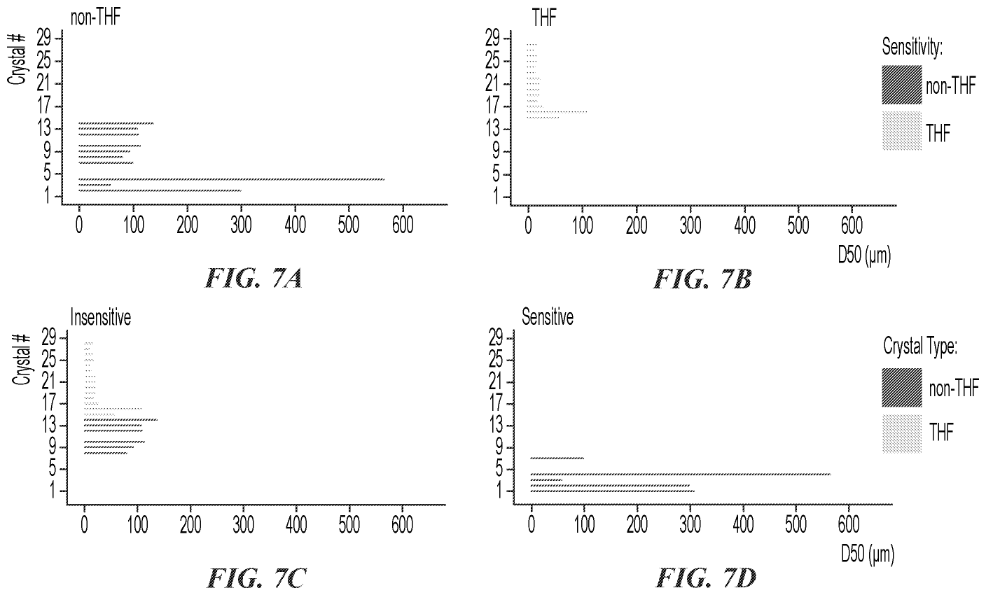

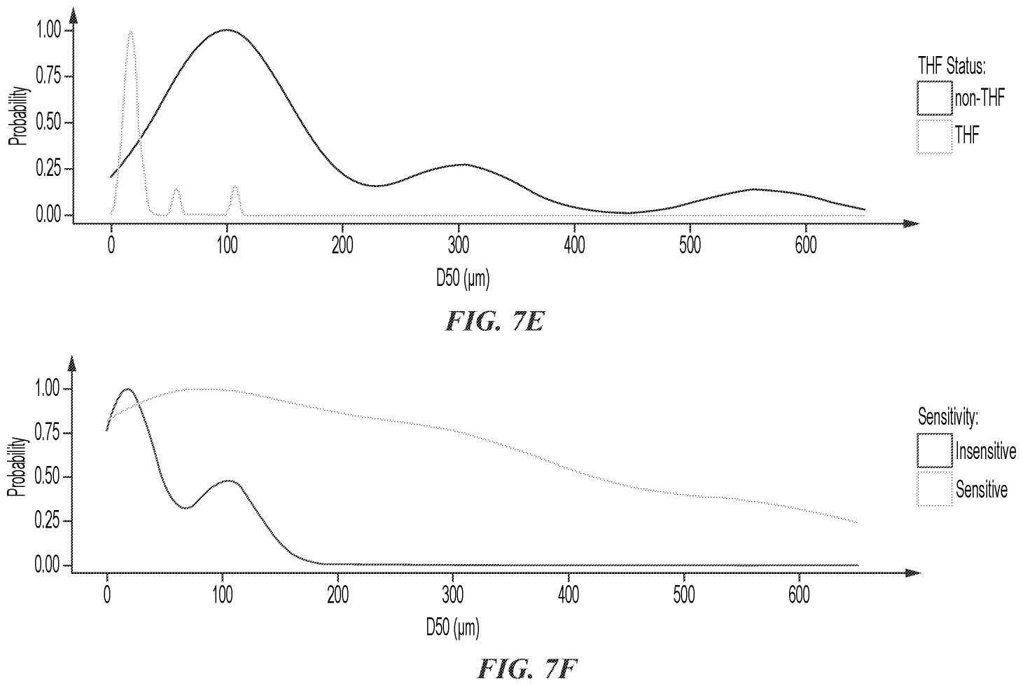

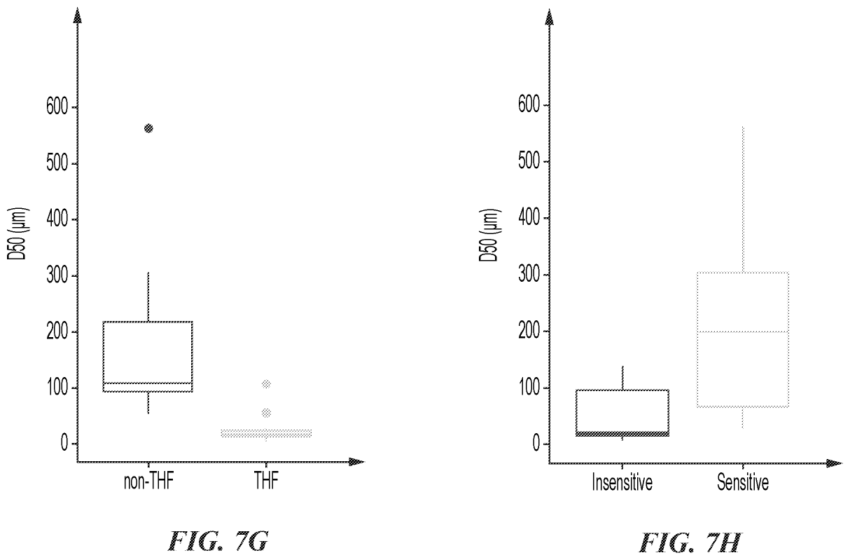

A graphical depiction of D50 measurements reveals an evident separation between THF-containing and THF free samples, as well as between impact sensitive and impact insensitive samples. FIG. 7A depicts D50 of RRx-001 particles crystalized without THF; FIG. 7B depicts D50 of RRx-001 particles crystalized with THF; FIG. 6C depicts D50 of impact insensitive RRx-001 particles; and FIG. 7D depicts D50 of impact sensitive RRx-001 particles.

The D50 empirical probability functions are provided in FIGS. 7E and 7F, and the boxplots are provided in FIGS. 7G and 7H. The visual separation is accompanied by a statistically significant findings based on the nonparametric Wilcoxon rank sum test with continuity correction applied to test the null hypothesis that the true D50 location shift is zero (i.e., equality of medians) against the alternative hypothesis that the true location shift is not zero. The Wilcoxon test identified a statistically significant difference between impact sensitive and insensitive particles (W statistic=20.5 and a corresponding two-sided p-value=0.0173) and also between THF-containing and THF-free particles (W statistic=160 and a p-value <0.0001). A Kolmogorov-Smirnov (KS) test comparing the distributions of the D50 also identified a statistically significant difference between impact sensitive and insensitive particles (KS W statistic=0.6, p=0.0720) and between THF-containing and THF-free particles (KS W statistic=0.8666 and p=0.0001).

### D90 Measurements Distributional Comparison

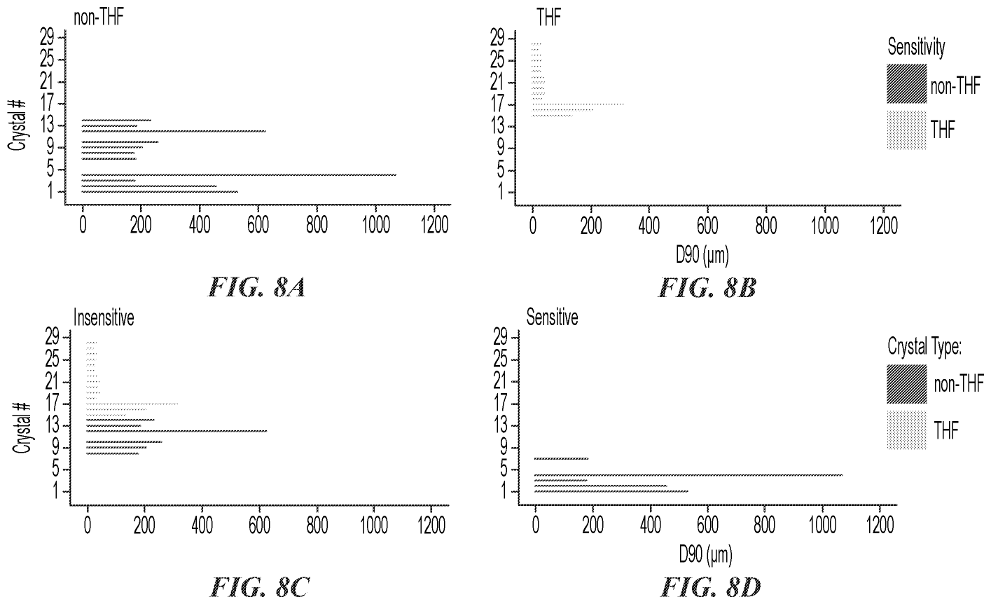

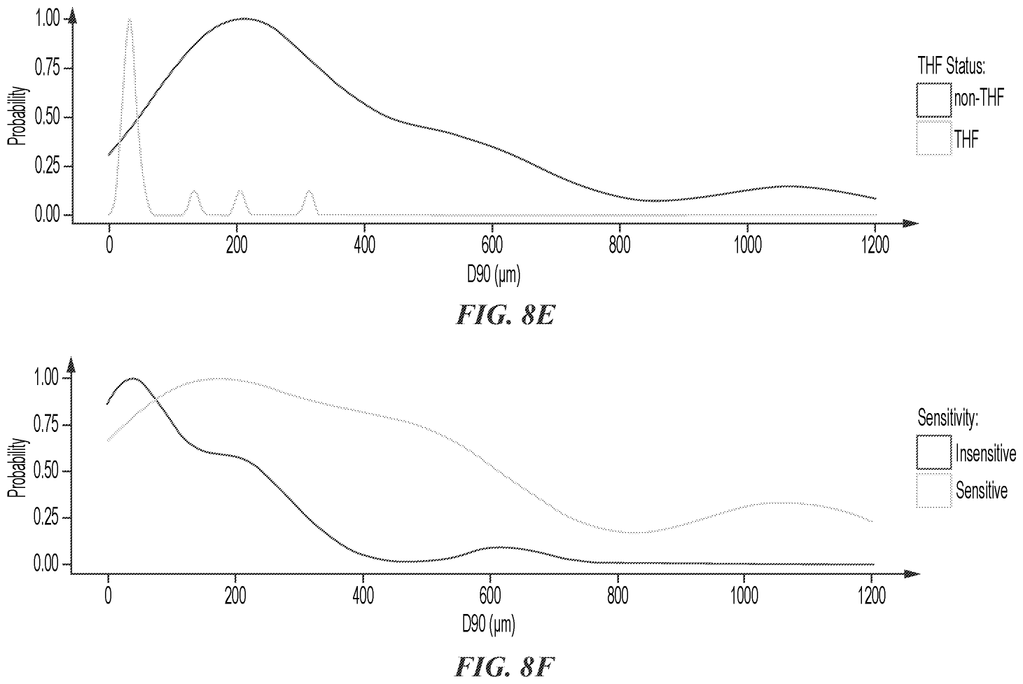

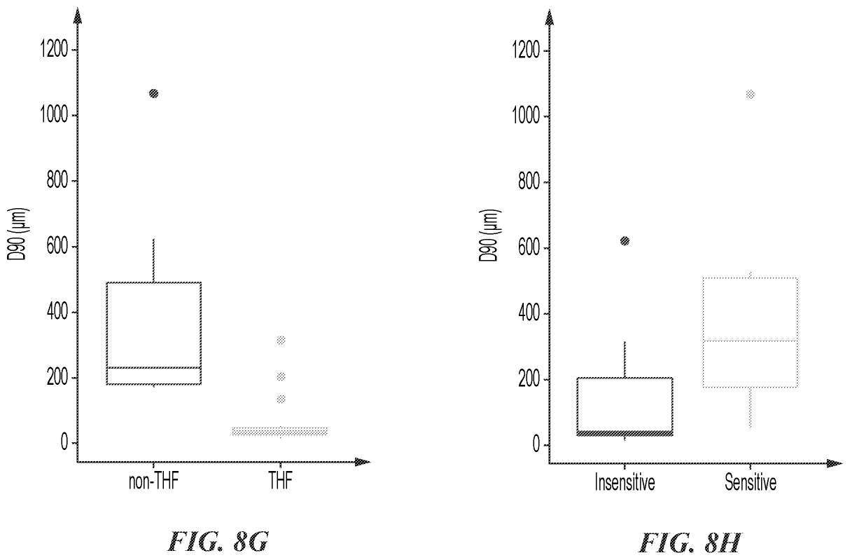

A graphical depiction of D90 measurements reveals an evident separation between THF-containing and THF free samples, as well as between impact sensitive and impact insensitive samples. FIG. 8A depicts D90 of RRx-001 particles crystalized without THF; FIG. 8B depicts D90 of RRx-001 particles crystalized with THF; FIG. 8C depicts D90 of impact insensitive RRx-001 particles; and FIG. 8D depicts D90 of impact sensitive RRx-001 particles.

The D90 empirical probability functions are provided in FIGS. 8E and 8F, and the boxplots are provided in FIGS. 8G and 8H. The visual separation is accompanied by a statistically significant findings based on the nonparametric Wilcoxon rank sum test with continuity correction applied to test the null hypothesis that the true D90 location shift is zero (i.e., equality of medians) against the alternative hypothesis that the true location shift is not zero. The Wilcoxon test identified a statistically significant difference between impact sensitive and insensitive particles (W statistic=25 and a corresponding two-sided p-value=0.0354) and also between THF-containing and THF-free particles (W statistic=153 and a p-value=0.0002). A Kolmogorov-Smirnov (KS) test comparing the distributions of the D90 also identified a statistically significant difference between impact sensitive and insensitive particles (KS W statistic=0.55, p=0.1225) and between THF-containing and THF-free particles (KS W statistic=0.8666 and p=0.0001).

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

We claim:

1. A composition comprising solid crystalline, impact- or detonation-insensitive particles comprising the compound of Formula I: 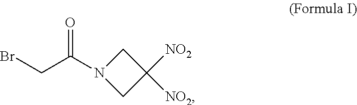 [SMILES: O=C(CBr)N1CC([N+](=O)[O-])([N+](=O)[O-])C1]
or a pharmaceutically acceptable salt thereof.

2. The composition of claim 1, wherein the composition comprises the compound in a solvated form.

3. The composition of claim 1 or 2, wherein the composition comprises tetrahydrofuran (THF).

4. The composition of claim 3, wherein the concentration of THF in the composition is at least about 330 ppm.

5. The composition of any one of claims 1-4, wherein the particles are in a clathrated form.

6. The composition of any one of claims 1-5, wherein the particles comprise THF.

7. The composition of claim 6, wherein the concentration of THF in the particles is at least about 330 ppm.

8. The composition of any one of claims 1-7, wherein the composition further comprises n-heptane.

9. The composition of claim 8, wherein the concentration of n-heptane in the composition is at least about 800 ppm.

10. The composition of any one of claims 1-9, wherein the particles comprise n-heptane.

11. The composition of claim 10, wherein the concentration of n-heptane in the particles is at least about 800 ppm.

12. The composition of any one of claims 1-11, wherein the composition has a bulk density in the range of from 0.1 g/cm³ to 0.6 g/cm³.

13. The composition of any one of claims 1-12, wherein the bulk density is from 0.15 g/cm³ to 0.5 g/cm³, from 0.15 g/cm³ to 0.4 g/cm³, or from 0.16 g/cm³ to 0.3 g/cm³.

14. The composition of any one of claims 1-13, wherein the particles have a bulk density less than about 0.45 g/cm³.

15. The composition of any one of claims 1-14, wherein Dv(10) of the particles is less than about 40 μm.

16. The composition of any one of claims 1-15, wherein Dv(50) of the particles is less than about 200 μm.

17. The composition of any one of claims 1-16, wherein Dv(90) of the particles is less than about 400 μm.

18. The composition of any one of claims 1-17, wherein the particles have a substantially needle-like shape.

19. The composition of any one of claims 1-18, wherein the composition has a solubility greater than about 20 mg/mL in DMSO at 25° C.

20. The composition of any one of claims 1-19, wherein the particles have an angle of repose of less than about 45 degrees.

21. The composition of any one of claims 1-20, wherein viability of cancer cells treated with the composition is lower than viability of cancer cells treated with an impact- or detonation-sensitive composition comprising equal amount of ABDNAZ.

22. The composition of any one of claims 1-21, wherein (i) viability of HCT 116 cells treated with about 8 μM ABDNAZ from the composition is at least about 50% lower than viability of HCT 116 cells treated with about 8 μM ABDNAZ from an impact- or detonation-sensitive composition; (ii) viability of SCC VII cells treated with about 4 μM ABDNAZ from the composition is at least about 25% lower than viability of SCC VII cells treated with about 4 μM ABDNAZ from an impact- or detonation-sensitive composition; or (iii) viability of A549 cells treated with about 20 μM ABDNAZ from the composition is at least about 25% lower than viability of A549 cells treated with about 20 μM ABDNAZ from an impact- or detonation-sensitive composition; as measured 24 hours after each treatment.

23. The composition of any one of claims 1-21, wherein (i) viability of HCT 116 cells treated with about 10 μM ABDNAZ from the composition is less than about 25% of viability of untreated HCT 116 cells; (ii) viability of SCC VII cells treated with about 4 μM ABDNAZ from the composition is less than about 50% of viability of untreated SCC VII cells; or (iii) viability of A549 cells treated with about 20 μM ABDNAZ from the composition is less than about 50% of viability of untreated A549 cells; as measured 24 hours after each treatment.

24. The composition of any one of claims 1-23, wherein the particles are dispersed in a dedusting agent.

25. The composition of claim 24, wherein the dedusting agent is polyethylene glycol.

26. A pharmaceutical composition comprising the composition of any one of claims 1-25, and a pharmaceutically acceptable carrier.

27. The pharmaceutical composition of claim 26, further comprising N,N-dimethylacetamide.

28. The pharmaceutical composition of claim 26 or 27, further comprising an anti-coagulant.

29. A mixture comprising the composition of any one of claims 1-25 or the pharmaceutical composition of any one of claims 26-28, and a blood sample.

30. The mixture of claim 29, wherein the blood sample has been harvested from a subject to be treated with the compound.

31. The mixture of claim 30, wherein the concentration of the compound of Formula I is from 0.1 mg/mL of blood to 10 mg/mL of blood.

32. A method of producing a crystalline form of the compound of Formula I, the method comprising the steps of:
(a) dissolving the compound of Formula I in tetrahydrofuran;
(b) adding the solution of step (a) to n-heptane with stirring; and
(c) cooling the solution produced by step (b), thereby to provide the crystalline form of Formula I.

33. The method of claim 32, wherein the THF solution produced in step (a) is combined with the n-heptane in step (b) at a ratio of between about 1:3 (v/v) and about 1:10 (v/v).

34. The method claim 32 or 33, wherein, during step (b), the adding occurs over a period between about 10 minutes and about 6 hours.

35. A crystalline form of the compound of Formula I produced by the method of any one of claims 32-34.

36. The composition of any one of claims 1-25, wherein the composition is impact insensitive as determined by using a Series 3 Type (a)(ii) Test as set forth in the United Nations Manual of Tests and Criteria, seventh edition, 2019 by exposing a 40 mm³ sample of the composition to 40 J of energy.

37. The crystalline form of the compound of claim 35, wherein the crystalline form is impact insensitive as determined by using a Series 3 Type (a)(ii) Test as set forth in the United Nations Manual of Tests and Criteria, seventh edition, 2019 by exposing a 40 mm³ sample of the crystalline form to 40 J of energy.

38. A method treating cancer in a subject in need thereof, the method comprising administering to the subject an effective amount of the composition of any one of claims 1-25, the pharmaceutical composition of any one of claims 26-28, or the mixture of any one of claims 29-31, thereby to treat the cancer in the subject.

39. The method of claim 38, wherein the composition of any one of claims 1-25 or the pharmaceutical composition of any one of claims 26-28 is combined with blood harvested from the subject to create a mixture, whereupon the mixture is administered to the subject.

40. A method of treating or preventing an ischemic or hypoxic condition in a subject in need thereof, the method comprising administering to the subject a therapeutically effective amount of the composition of any one of claims 1-25 or a pharmaceutical composition of any one of claims 26-28 or a mixture of any one of claims 29-31.

41. The method of claim 40, wherein the ischemic condition is an acute or chronic ischemic condition.

42. The method of claim 41, wherein the acute ischemic condition is myocardial infarction, ischemic stroke, pulmonary embolism, perinatal hypoxia, circulatory shock, mountain sickness or acute respiratory failure.

43. The method of claim 41, wherein the chronic ischemic condition is atherosclerosis, chronic venous insufficiency, chronic heart failure, cardiac cirrhosis, diabetes, macular degeneration, sleep apnea, Raynaud's disease, systemic sclerosis, nonbacterial thrombotic endocarditis, occlusive artery disease, angina pectoris, transient ischemic attacks, or chronic alcoholic liver disease.

44. The method of claim 40, wherein the hypoxic condition is cancer, gastric or duodenal ulcers, liver or renal disease, thrombocytopenia, a blood coagulation disorder, a chronic illness, a therapeutic intervention that produces anemia such as cancer chemotherapy or altitude sickness.

45. The method of claim 44, wherein the cancer is bladder cancer, breast cancer, clear cell kidney cancer, head/neck squamous cell carcinoma, lung squamous cell carcinoma, malignant melanoma, colorectal cancer, head and neck cancer, cervical cancer, non-small-cell lung cancer (NSCLC), ovarian cancer, pancreatic cancer, prostate cancer, renal cell cancer, small-cell lung cancer (SCLC), triple negative breast cancer, acute lymphoblastic leukemia (ALL), acute myeloid leukemia (AML), chronic lymphocytic leukemia (CLL), chronic myeloid leukemia (CML), diffuse large B-cell lymphoma (DLBCL), EBV-positive DLBCL, primary mediastinal large B-cell lymphoma, T-cell/histiocyte-rich large B-cell lymphoma, follicular lymphoma, Hodgkin's lymphoma (HL), mantle cell lymphoma (MCL), multiple myeloma (MM), myeloid cell leukemia-1 protein (Mcl-1), myelodysplasia syndrome (MDS), non-Hodgkin's lymphoma (NHL), or small lymphocytic lymphoma (SLL).

46. The method of any one of claims 38-45, wherein the pharmaceutical composition comprises at least 0.5 mg of the compound of Formula I and is administered intravenously, nasally, optically, intraperitoneally, subcutaneously, or orally.

47. A method of protecting against normal tissue toxicity caused by chemotherapy and/or radiation therapy, the method comprising: subcutaneously administering to a subject in need thereof an effective amount of the composition of any one of claims 1-25, the pharmaceutical composition of any one of claims 26-28, or the mixture of any one of claims 29-31 before the subject is exposed to the chemotherapy and/or radiation therapy.

48. The method of claim 47, wherein the subject has cancer.

49. The method of claim 48, wherein the cancer is head and neck cancer.

50. The method of any one of claims 47-49, wherein at least about 0.5 mg of the compound of Formula I is administered to the subject.

51. The method of claim 50, wherein from about 0.5 mg to 4 mg of the compound of Formula I is administered to the subject.

52. The method of claim 50 or 51, wherein the amount of the compound of Formula I is administered in one or more divided injections.

53. The method of any one of claims 47-52, wherein the normal tissue toxicity is acute mucositis or dysphagia.

54. The method of claim 53, wherein the mucositis is late mucositis.

55. A method of treating a disorder selected from the group consisting of an autoimmune disorder, inflammatory disorder, neurodegenerative disorder, and neuromuscular disorder in a subject in need thereof, the method comprising administering a loading dose of the composition of any one of claims 1-25, the pharmaceutical composition of any one of claims 26-28, or the mixture of any one of claims 29-31 to the subject in an amount effective to ameliorate a symptom of the disorder, and thereafter administering a maintenance dose of the composition of any one of claims 1-25, the pharmaceutical composition of any one of claims 26-28, or the mixture of any one of claims 29-31 to maintain the amelioration of the symptom for a prolonged period of time.

56. A method for increasing compliance and tolerability in a subject in need of treatment for an autoimmune disorder, inflammatory disorder, neurodegenerative disorder, or neuromuscular disorder, the method comprising administering a therapeutically effective amount of the composition of any one of claims 1-25, the pharmaceutical composition of any one of claims 26-28, or the mixture of any one of claims 29-31; wherein administration of the therapeutically effective amount does not cause hematologic, neurologic, pulmonary, metabolic, cardiovascular, dermatologic, nephrologic, gastrointestinal, genitourinary, inflammatory, autoimmune, thyroidal, and immunodeficiency-related side effects; and wherein the subject completes treatment with a cumulative dose of at least 1 mg or 1 mg/m² of RRx-001 or an analog thereof.

57. A method of preventing the initiation, development or worsening of a symptom of a disorder selected from the group consisting of an autoimmune disorder, inflammatory disorder, neurodegenerative disorder, and neuromuscular disorder in a subject in need thereof, the method comprising administering an effective amount of the composition of any one of claims 1-25, the pharmaceutical composition of any one of claims 26-28, or the mixture of any one of claims 29-31 to the subject to prevent the initiation, development, or worsening of the symptom of the disorder.

58. A method of preventing the initiation, development or worsening of a symptom of a disorder selected from the group consisting of an autoimmune disorder, inflammatory disorder, neurodegenerative disorder, and neuromuscular disorder in a subject in need thereof, the method comprising administering an effective amount of the composition of any one of claims 1-25, the pharmaceutical composition of any one of claims 26-28, or the mixture of any one of claims 29-31 to the subject to prevent the initiation, development, or worsening of the symptom of the disorder.

59. A method for enhancing physical performance of a mammal, the method comprising: administering an effective amount of the composition of any one of claims 1-25, the pharmaceutical composition of any one of claims 26-28, or the mixture of any one of claims 29-31 to said mammal prior to said physical performance.

60. A method for preventing or treating pulmonary hypertension (PH) in a patient, the method comprising: administering a therapeutically effective amount of the composition of any one of claims 1-25, the pharmaceutical composition of any one of claims 26-28, or the mixture of any one of claims 29-31.
